(19) United States
(12) Patent Application Publication
(10) Pub. No.: US 2018/0092628 A1
(43) Pub. Date: Apr. 5, 2018

(54) ULTRASONIC DIAGNOSTIC APPARATUS

(71) Applicant: Toshiba Medical Systems Corporation, Otawara-shi (JP)

(72) Inventors: Yoshitaka MINE, Nasushiobara (JP); Satoshi Matsunaga, Nasushiobara (JP); Yukifumi Kobayashi, Yokohama (JP); Kazuo Tezuka, Nasushiobara (JP); Jiro Higuchi, Otawara (JP); Atsushi Nakai, Nasushiobara (JP); Shigemitsu Nakaya, Nasushiobara (JP); Yutaka Kobayashi, Nasushiobara (JP)

(73) Assignee: Toshiba Medical Systems Corporation, Otawara-shi (JP)

(21) Appl. No.: 15/718,578

(22) Filed: Sep. 28, 2017

(30) Foreign Application Priority Data
Sep. 30, 2016 (JP) 2016-195129

(51) Int. Cl. A61B 8/00

(52) U.S. Cl. CPC A61B 8/463; A61B 8/13; A61B 8/465; A61B 8/466

(57) ABSTRACT

According to one embodiment, an ultrasonic diagnostic apparatus includes processing circuitry. The processing circuitry acquires position information relating to an ultrasonic probe and an ultrasonic image. The processing circuitry acquires ultrasonic image data which is obtained by transmission and reception of ultrasonic from the ultrasonic probe at a position where the position information is acquired, the ultrasonic image data being associated with the position information. The processing circuitry executes associating between a first coordinate system relating to the position information and a second coordinate system relating to medical image data. The processing circuitry executes image alignment between an ultrasonic image based on the associated ultrasonic image data and a medical image based on the medical image data.

### CROSS-REFERENCE TO RELATED APPLICATIONS

This application is based upon and claims the benefit of priority from the prior Japanese Patent Application No. 2016-195129, filed Sep. 30, 2016, the entire contents of all of which are incorporated herein by reference.

### FIELD

Embodiments described herein relate generally to an ultrasonic diagnostic apparatus.

### BACKGROUND

In recent years, in medical image diagnosis, alignment between three-dimensional image data, which are acquired by using a medical image diagnostic apparatus (an X-ray computer tomography apparatus, a magnetic resonance imaging apparatus, an ultrasonic diagnostic apparatus, an X-ray diagnostic apparatus, a nuclear medical diagnostic apparatus, etc.), has been performed by using various methods.

For example, alignment between three-dimensional (3D) ultrasonic image data and other three-dimensional (3D) medical image data is performed by acquiring, with use of an ultrasonic probe to which a position sensor is attached, three-dimensional image data to which position information is added, and by using this position information and position information which is added to the other 3D medical image data.

Besides, alignment between three-dimensional CT (Computed Tomography) image data and three-dimensional MR (magnetic resonance) image data is performed by analyzing the respective image data, specifying a region which functions as a landmark, and making the specified regions correspond to each other.

### BRIEF DESCRIPTION OF THE DRAWING

FIG. 1 is a block diagram illustrating an ultrasonic diagnostic apparatus according to a present embodiment.

FIG. 2 is a conceptual view illustrating three-dimensional display of ultrasonic image data.

FIG. 3 is a flowchart illustrating an alignment process between ultrasonic image data.

FIG. 4 is a flowchart illustrating an image alignment process.

FIG. 5 is a view illustrating an example of ultrasonic image display before alignment between ultrasonic image data.

FIG. 6 is a view illustrating an example of ultrasonic image display after the alignment between the ultrasonic image data.

FIG. 7 is a flowchart illustrating an alignment process between ultrasonic image data according to a second embodiment.

FIG. 8 is a view illustrating an example of ultrasonic image display after completion of sensor alignment.

FIG. 9 is a flowchart illustrating an alignment process between ultrasonic image data and medical image data.

FIG. 10A is a conceptual view of sensor alignment between ultrasonic image data and medical image data.

FIG. 10B is a conceptual view of sensor alignment between ultrasonic image data and medical image data.

FIG. 10C is a conceptual view of sensor alignment between ultrasonic image data and medical image data.

FIG. 11A is a schematic view of an example of a case in which a doctor conducts an examination of the liver.

FIG. 11B is a view illustrating an example in which ultrasonic image data and medical image data are associated.

FIG. 12 is a view for describing correction of displacement between ultrasonic image data and medical image data.

FIG. 13 is a view illustrating an example of acquisition of ultrasonic image data in a state in which the correction of displacement is completed.

FIG. 14 is a view illustrating an example of ultrasonic image display after alignment between ultrasonic image data and medical image data.

FIG. 15 is a view illustrating an example of synchronous display between an ultrasonic image and a medical image.

FIG. 16 is a view illustrating another example of synchronous display between an ultrasonic image and a medical image.

FIG. 17 is a flowchart illustrating another example of an alignment process between ultrasonic image data and medical image data.

FIG. 18 is a view illustrating a display example before alignment between ultrasonic image data and medical image data.

FIG. 19 is a view illustrating a display example after alignment between ultrasonic image data and medical image data.

FIG. 20 is a view illustrating another display example after alignment between ultrasonic image data and medical image data.

FIG. 21 is a block diagram illustrating an ultrasonic diagnostic apparatus in a case of utilizing infrared for a position sensor system.

FIG. 22 is a block diagram illustrating an ultrasonic diagnostic apparatus in a case of utilizing robotic arms for a position sensor system.

FIG. 23 is a block diagram illustrating an ultrasonic diagnostic apparatus in a case of utilizing a gyro sensor for a position sensor system.

FIG. 24 is a block diagram illustrating an ultrasonic diagnostic apparatus in a case of utilizing a camera for a position sensor system.

FIG. 25 is a conceptual view illustrating a position sensor system by a magnetic sensor.

FIG. 26 is a conceptual view illustrating a position sensor system by a magnetic sensor, in a case in which a living body has moved during an ultrasonic examination.

FIG. 27 is a conceptual view illustrating a position sensor system in a case of disposing a magnetic sensor on a body surface.

FIG. 28 is a conceptual view of an ultrasonic diagnostic apparatus, illustrating an operation example of a position sensor-equipped 2D array probe.

FIG. 29 is a view illustrating a flow of a process of real-time 3D alignment display.

FIG. 30 is a view illustrating a common structure between medical images.

FIG. 31 is a view illustrating an example of display of, an alignment quality between 3D ultrasonic image data.

FIG. 32 is a view illustrating an example of display of an alignment quality between 3D medical image data and 3D ultrasonic image data.

FIG. 33 is a flowchart illustrating another example of the image alignment process.

FIG. 34 is a view illustrating an example of a process of excluding a noise region of 3D ultrasonic image data.

FIG. 35 is a view illustrating an example of a process of extracting a blood vessel structure by 3D ultrasonic color data.

### DETAILED DESCRIPTION

There are the following problems in the alignment between the 3D ultrasonic image data and 3D medical image data (three-dimensional image data of CT or MR, which is acquired by a medical image diagnostic apparatus) by the conventional method.

To begin with, an alignment operation with a CT or MR image has to be performed by a manual technique with an ultrasonic probe. Thus, a displacement occurs mainly in angular components, and the precision in alignment in the entirety of a region-of-interest tends to lower. In addition, it depends on the user's skill to perform alignment by finding in the 3D ultrasonic image data a structure common to the CT image or MR image. Thus, a variance occurs in precision of alignment. A tissue, a blood vessel, or blood appears differently between the CT image or MR image, and the ultrasonic image. In the case of ultrasonic, a structure relating to a gas or a deep portion of a bone cannot be viewed. In addition, an ultrasonic image of 3D display has a very small volume region, compared to the CT or MR. Thus, only a part of the structure is included in the ultrasonic image.

In the CT or MR, the direction of an image is kept constant by the bed. However, the direction of the image of 3D ultrasonic image data is freely variable, depending on how to apply the ultrasonic probe. Thus, in the alignment with the CT image or MR image, both the positional displacement and the angular displacement increase, and it is necessary to set a wide search range for alignment. However, if the search range is set to be large, it is highly possible that the ultrasonic image is trapped at a local optimal point and alignment fails to be achieved, and the success rate decreases. Accordingly, there is a difficulty in performing image alignment between the CT or MR image and the ultrasonic image. In research organizations or ultrasonic diagnostic apparatuses, attempts have been made to perform image alignment between the CT or MR image and the ultrasonic image, but these attempts are unsuccessful, and the quality in practical use is not secured. In ultrasonic diagnostic apparatuses, diagnosis is mostly conducted by two-dimensional tomographic images, and 3D ultrasonic image data is scarcely present, and this leads to a hindrance to alignment between the CT or MR image and the ultrasonic image. Furthermore, when alignment between 3D ultrasonic image data is considered, the alignment becomes alignment between small volumes, and the degree of freedom in position or direction is large, resulting in difficulty in securing overlap between data. A small overlap means that the number of included common structures is small. The image alignment between 3D ultrasonic image data has not been widely researched, and this alignment has not been put to practical use.

From the above points, the success rate of the image alignment between the 3D ultrasonic image data and the 3D medical image data by the conventional methods is low, and it can be said that the image alignment between the 3D ultrasonic image data and the 3D medical image data by the conventional methods is not practical.

In general, according to one embodiment, an ultrasonic diagnostic apparatus includes processing circuitry. The processing circuitry acquires position information relating to an ultrasonic probe and an ultrasonic image. The processing circuitry acquires ultrasonic image data which is obtained by transmission and reception of ultrasonic from the ultrasonic probe at a position where the position information is acquired, the ultrasonic image data being associated with the position information. The processing circuitry executes associating between a first coordinate system relating to the position information and a second coordinate system relating to medical image data. The processing circuitry executes image alignment between an ultrasonic image based on the associated ultrasonic image data and a medical image based on the medical image data.

Hereinafter, an ultrasonic diagnostic apparatus and an ultrasonic diagnosis support program according to embodiments will be described with reference to the accompanying drawings. In the embodiments to be described below, it is assumed that the parts denoted by like reference numerals perform the same operations, and overlapping descriptions will be omitted as needed.

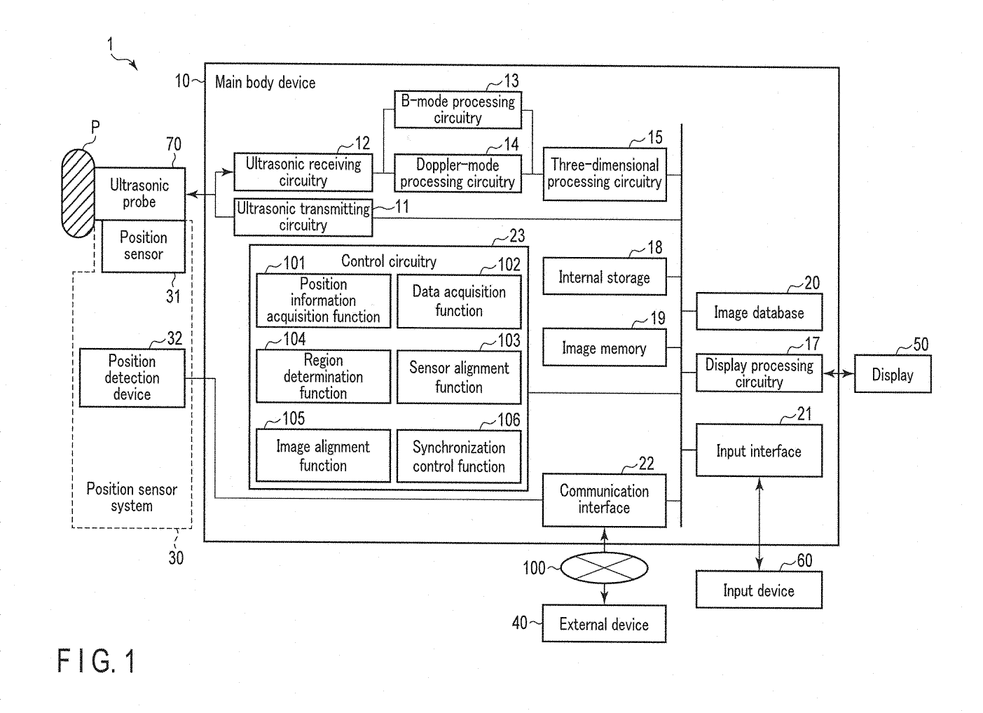

FIG. 1 is a block diagram illustrating a configuration example of an ultrasonic diagnostic apparatus 1 according to an embodiment. As illustrated in FIG. 1, the ultrasonic diagnostic apparatus 1 includes a main body device 10, an ultrasonic probe 70, and a position sensor system 30. The main body device 10 is connected to an external device 40 via a network 100. In addition, the main body device 10 is connected to a display 50 and an input device 60.

The position sensor system 30 is a system for acquiring three-dimensional position information of the ultrasonic probe 70 and an ultrasonic image. The position sensor system 30 includes a position sensor 31 and a position detection device 32.

The position sensor system 30 acquires three-dimensional position information of the ultrasonic probe 70 by attaching, for example, a magnetic sensor, an infrared sensor or a target for an infrared camera, as the position sensor 31 to the ultrasonic probe 70. A gyro sensor (angular velocity sensor) may be built in the ultrasonic probe 70, and this gyro sensor may acquire the three-dimensional position information of the ultrasonic probe 70. In addition, the position sensor system 30 may photograph the ultrasonic probe 70 by a camera, and may subject the photographed image to an image recognition process, thereby acquiring the three-dimensional position information of the ultrasonic probe 70. The position sensor system 30 may hold the ultrasonic probe 70 by robotic arms, and may acquire the position of the robotic arms in the three-dimensional space as the position information of the ultrasonic probe 70.

In the description below, a case is described, by way of example, in which the position sensor system 30 acquires position information of the ultrasonic probe 70 by using the magnetic sensor. Specifically, the position sensor system 30 further includes a magnetism generator (not shown) including, for example, a magnetism generating coil. The magnetism generator forms a magnetic field toward the outside, with the magnetism generator itself being set as the center. A magnetic field space, in which position precision is ensured, is defined in the formed magnetic field. Thus, it should suffice if the magnetism generator is disposed such that a living body, which is a target of an ultrasonic examination, is included in the magnetic field space in which position precision is ensured. The position sensor 31, which is attached to the ultrasonic probe 70, detects a strength and a gradient of a three-dimensional magnetic field which is formed by the magnetism generator. Thereby, the position and direction of the ultrasonic probe 70 are acquired. The position sensor 31 outputs the detected strength and gradient of the magnetic field to the position detection device 32.

The position detection device 32 calculates, based on the strength and gradient of the magnetic field which were detected by the position sensor 31, for example, a position of the ultrasonic probe 70 (a position (x, y, z) and a rotational angle (θx, θy, θz) of a scan plane) in a three-dimensional space with the origin set at a predetermined position. At this time, the predetermined position is, for example, a position where the magnetism generator is disposed. The position detection device 32 transmits position information relating to the calculated position (x, y, z, θx, θy, θz) to the main body device 10.

In the meantime, the position information can be imparted to the ultrasonic image data by associating, by time synchronization or the like, the position information acquired as described above and the ultrasonic image data of the ultrasonic which is transmitted and received by the ultrasonic probe 70.

The ultrasonic probe 70 includes a plurality of piezoelectric transducers, a matching layer provided on the piezoelectric transducers, and a backing material for preventing the ultrasonic waves from propagating backward from the piezoelectric transducers. The ultrasonic probe 70 is detachably connected to the main body device 10. Each of the plurality of piezoelectric transducers generates an ultrasonic wave based on a driving signal supplied from ultrasonic transmission circuitry 11 included in the main body device 10. In addition, buttons, which are pressed at a time of an offset process (to be described later), at a time of a freeze of an ultrasonic image, and the like, may be disposed on the ultrasonic probe 70.

When the ultrasonic probe 70 transmits ultrasonic waves to a living body P, the transmitted ultrasonic waves are sequentially reflected by a discontinuity surface of acoustic impedance of the living tissue of the living body P, and received by the plurality of piezoelectric transducers of the ultrasonic probe 70 as a reflected wave signal. The amplitude of the received reflected wave signal depends on an acoustic impedance difference on the discontinuity surface by which the ultrasonic waves are reflected. Note that the frequency of the reflected wave signal generated when the transmitted ultrasonic pulses are reflected by moving blood or the surface of a cardiac wall or the like shifts depending on the velocity component of the moving body in the ultrasonic transmission direction due to the Doppler effect. The ultrasonic probe 70 receives the reflected wave signal from the living body P, and converts it into an electrical signal.

As described above, since the position sensor 31 is attached to the ultrasonic probe 70 according to the present embodiment, the position information at a time when the ultrasonic probe 70 three-dimensionally scans the living body P can be detected. Specifically, the ultrasonic probe 70 according to the present embodiment is a one-dimensional array probe including a plurality of ultrasonic transducers which two-dimensionally scans the living body P. In the meantime, the ultrasonic probe 70, to which the position sensor 31 is attached, may be a mechanical four-dimensional probe (a three-dimensional probe of a mechanical swing method) which is configured such that a one-dimensional array probe and a motor for swinging the probe are provided in a certain enclosure, and ultrasonic transducers are swung at a predetermined angle (swing angle). Thereby, a tilt scan or rotational scan is mechanically performed, and the living body P is three-dimensionally scanned. Besides, the ultrasonic probe 70 may be a two-dimensional array probe in which a plurality of ultrasonic transducers are arranged in a matrix, or a 1.5-dimensional array probe in which a plurality of transducers that are one-dimensionally arranged are divided into plural parts.

The main body device 10 illustrated in FIG. 1 is an apparatus which generates an ultrasonic image, based on the reflected wave signal which the ultrasonic probe 70 receives. As illustrated in FIG. 1, the main body device 10 includes the ultrasonic transmitting circuitry 11, ultrasonic receiving circuitry 12, B-mode processing circuitry 13, Doppler-mode processing circuitry 14, three-dimensional processing circuitry 15, display processing circuitry 17, an internal storage 18, an image memory 19 (cine memory), an image database 20, input interface 21, communication interface 22, and control circuitry 23.

The ultrasonic transmitting circuitry 11 is a processor which supplies a driving signal to the ultrasonic probe 70. The ultrasonic transmitting circuitry 11 is realized by, for example, trigger generating circuitry, delay circuitry, and pulser circuitry. The trigger generating circuitry repeatedly generates, at a predetermined rate frequency, rate pulses for forming transmission ultrasonic. The delay circuitry imparts, to each rate pulse generated by the trigger generating circuitry, a delay time for each piezoelectric transducer which is necessary for determining transmission directivity by converging ultrasonic, which is generated from the ultrasonic probe 70, into a beam form. The pulser circuitry applies a driving signal (driving pulse) to the ultrasonic probe 70 at a timing based on the rate pulse. By varying the delay time that is imparted to each rate pulse by the delay circuitry, the transmission direction from the piezoelectric transducer surface can arbitrarily be adjusted.

The ultrasonic receiving circuitry 12 is a processor which executes various processes on the reflected wave signal which the ultrasonic probe 70 receives, and generates a reception signal. The ultrasonic receiving circuitry 12 is realized by, for example, amplifier circuitry, an A/D converter, reception delay circuitry, and an adder. The amplifier circuitry executes a gain correction process by amplifying, on a channel-by-channel basis, the reflected wave signal which the ultrasonic probe 70 receives. The A/D converter converts the gain-corrected reflected wave signal to a digital signal. The reception delay circuitry imparts a delay time, which is necessary for determining reception directivity, to the digital signal. The adder adds a plurality of digital signals to which the delay time was imparted. By the addition process of the adder, a reception signal is generated in which a reflected component from a direction corresponding to the reception directivity is emphasized.

The B-mode processing circuitry 13 is a processor which generates B-mode data, based on the reception signal received from the ultrasonic receiving circuitry 12. The B-mode processing circuitry 13 executes an envelope detection process and a logarithmic amplification process on the reception signal received from the ultrasonic receiving circuitry 12, and generates data (B-mode data) in which the signal strength is expressed by the magnitude of brightness. The generated B-mode data is stored in a RAW data memory (not shown) as B-mode RAW data on a two-dimensional ultrasonic scanning line.

The Doppler-mode processing circuitry 14 is a processor which generates a Doppler waveform and Doppler data, based on the reception signal received from the ultrasonic receiving circuitry 12. The Doppler-mode processing circuitry 14 extracts a blood flow signal from the reception signal, generates a Doppler waveform from the extracted blood flow signal, and generates data (Doppler data) in which information, such as a mean velocity, dispersion and power, is extracted from the blood flow signal with respect to multiple points.

The three-dimensional processing circuitry 15 is a processor which can generate three-dimensional image data with position information, based on the data generated by the B-mode processing circuitry 13 and the Doppler-mode processing circuitry 14. When the ultrasonic probe 70, to which the position sensor 31 is attached, is the one-dimensional array probe or 1.5-dimensional array probe, the three-dimensional processing circuitry 15 adds the position information of the ultrasonic probe 70, which is calculated by the position detection device 32, to the B-mode RAW data stored in the RAW data memory. In addition, the three-dimensional processing circuitry 15 may generate two-dimensional image data which is composed of pixels, by executing RAW-pixel conversion, and may add the position information of the ultrasonic probe 70, which is calculated by the position detection device 32, to the generated two-dimensional image data.

Furthermore, the three-dimensional processing circuitry 15 generates three-dimensional image data (hereinafter referred to as “volume data”) which is composed of voxels in a desired range, by executing RAW-voxel conversion, which includes an interpolation process with spatial position information being taken into account, on the B-mode RAW data stored in the RAW data memory. The position information of the ultrasonic probe 70, which is calculated by the position detection device 32, is added to the volume data. Similarly, when the ultrasonic probe 70, to which the position sensor 31 is attached, is the mechanical four-dimensional probe (three-dimensional probe of the mechanical swing method) or the two-dimensional array probe, the position information is added to the two-dimensional RAW data, two-dimensional image data and three-dimensional image data.

The three-dimensional processing circuitry 15 generates rendering image data by applying a rendering process to the generated volume data.

The display processing circuitry 17 executes various processes, such as dynamic range, brightness, contrast and y curve corrections, and RGB conversion, on various image data generated in the three-dimensional processing circuitry 15, thereby converting the image data to a video signal. The display processing circuitry 17 causes the display 50 to display the video signal. In the meantime, the display processing circuitry 17 may generate a user interface (GUI: Graphical User Interface) for an operator to input various instructions by the input interface 21, and may cause the display 50 to display the GUI. For example, a CRT display, a liquid crystal display, an organic EL display, an LED display, a plasma display, or other arbitrary display known in the present technical field, may be used as needed as the display 50.

The internal storage 18 includes, for example, a storage medium which can be read by a processor, such as a magnetic or optical storage medium, or a semiconductor memory. The internal storage 18 stores a control program for realizing ultrasonic transmission/reception, a control program for executing an image process, and a control program for executing a display process. In addition, the internal storage 18 stores diagnosis information (e.g. patient ID, doctor's findings, etc.), a diagnosis protocol, a body mark generation program, and data such as a conversion table for presetting a range of color data for use in imaging, with respect to each of regions of diagnosis. Besides, the internal storage 18 may store anatomical illustrations, for example, an atlas, relating to the structures of internal organs in the body.

In addition, the internal storage 18 stores two-dimensional image data, volume data and rendering image data which were generated by the three-dimensional processing circuitry 15, in accordance with a storing operation which is input via the input interface 21. Furthermore, in accordance with a storing operation which is input via the input interface 21, the internal storage 18 may store two-dimensional image data with position information, volume data with position information and rendering image data with position information which were generated by the three-dimensional processing circuitry 15, along with the order of operations and the times of operations. The internal storage 18 can transfer the stored data to an external device via the communication interface 22.

The image memory 19 includes, for example, a storage medium which can be read by a processor, such as a magnetic or optical storage medium, or a semiconductor memory. The image memory 19 stores image data corresponding to a plurality of frames immediately before a freeze operation which is input via the input interface 21. The image data stored in the image memory 19 is, for example, successively displayed (cine-displayed).

The image database 20 stores image data which is transferred from the external device 40. For example, the image database 20 acquires, from the external device 40, past image data relating to the same patient, which was acquired in past diagnosis, and stores the past image data. The past image data includes ultrasonic image data, CT (Computed Tomography) image data, MR image data, PET (Positron Emission Tomography)-CT image data, PET-MR image data, and X-ray image data.

The image database 20 may store desired image data by reading in image data which is stored in storage media such as an MO, CD-R and DVD.

The input interface 21 accepts various instructions from the user via the input device 60. The input device 60 is, for example, a mouse, a keyboard, a panel switch, a slider switch, a trackball, a rotary encoder, an operation panel, and a touch command screen (TCS). The input interface 21 is connected to the control circuitry 23, for example, via a bus, converts an operation instruction, which is input from the operator, to an electric signal, and outputs the electric signal to the control circuitry 23. In the present specification, the input interface 21 is not limited to input interface which is connected to physical operation components such as a mouse and a keyboard. Examples of the input interface 21 include processing circuitry of an electric signal, which receives, as a wireless signal, an electric signal corresponding to an operation instruction that is input from an external input device provided separately from the ultrasonic diagnostic apparatus 1, and outputs this electric signal to the control circuitry 23.

The communication interface 22 is connected, for example, wirelessly, to the position sensor system 30, and receives position information which is transmitted from the position detection device 32. In addition, the communication interface 22 is connected to the external device 40 via the network 100 or the like, and executes data communication with the external device 40. The external device 40 is, for example, a database of a PACS (Picture Archiving and Communication System) which is a system for managing the data of various kinds of medical images, or a database of an electronic medical record system for managing electronic medical records to which medical images are added. In addition, the external device 40 is, for example, various kinds of medical image diagnostic apparatuses other than the ultrasonic diagnostic apparatus 1 according to the present embodiment, such as an X-ray CT apparatus, an MRI (Magnetic Resonance Imaging) apparatus, a nuclear medical diagnostic apparatus, and an X-ray diagnostic apparatus. In the meantime, the standard of communication with the external device 40 may be any standard. An example of the standard is DICOM (digital imaging and communication in medicine).

The control circuitry 23 is, for example, a processor which functions as a central unit of the ultrasonic diagnostic apparatus 1. The control circuitry 23 executes a control program which is stored in the internal storage, thereby realizing functions corresponding to this program. Specifically, the control circuitry 23 executes a position information acquisition function 101, a data acquisition function 102, a sensor alignment function 103, a region determination function 104, an image alignment function 105, and a synchronization control function 106.

By executing the position information acquisition function 101, the control circuitry 23 acquires position information relating to the ultrasonic probe 70 from the position sensor system 30 via the communication interface 22.

By executing the data acquisition function 102, the control circuitry 23 acquires ultrasonic image data from the three-dimensional processing circuitry 15, and generates ultrasonic image data with position information, by associating the ultrasonic image data and the position information.

By executing the sensor alignment function 103, the control circuitry 23 associates the coordinate system of the position sensor and the coordinate system of 3D medical image data. As regards the ultrasonic image data, after the position information is defined by the position sensor coordinate system, the ultrasonic image data with position information and the 3D medical image data are aligned. The sensor alignment function 103 is an alignment function of alignment between 3D medical images in the sensor coordinate system. The ultrasonic image data is data of a free direction and position between a 3D medical image and a 3D ultrasonic image, or between 3D ultrasonic images. Thus, it is necessary to increase the search range for image alignment. However, by executing alignment in the coordinate system of the position sensor by the sensor alignment function 103, it is possible to perform rough adjustment of alignment between 3D medical image data. In the state in which the difference in position and rotation between the 3D medical image data is decreased, the image alignment that is the next step can be performed. In other words, the sensor alignment has a function of suppressing the difference in position and rotation between the 3D medical image data within a capture range of an image alignment algorithm.

By executing the region determination function 104, the control circuitry 23 receives, for example, an input to the input device 60 from the user via the input interface 21, and determines, based on the input, region information which serves as a reference for image alignment in at least one of the ultrasonic image and medical image.

By executing the image alignment function 105, the control circuitry 23 executes image alignment between an ultrasonic image based on the ultrasonic image data and a medical image based on the medical image data, the ultrasonic image data and medical image data being associated by the sensor alignment function 103.

By executing the synchronization control function 106, the control circuitry 23 synchronizes, based on the relationship between a first coordinate system and a second coordinate system, which was determined by the completion of the image alignment, a real-time ultrasonic image, which is an image based on ultrasonic image data newly acquired by the ultrasonic probe 70, and a medical image based on medical image data corresponding to the real-time ultrasonic image, and displays the real-time ultrasonic image and the medical image in an interlocking manner.

The position information acquisition function 101, data acquisition function 102, sensor alignment function 103, region determination function 104, image alignment function 105 and synchronization control function 106 may be assembled as the control program. Alternatively, dedicated hardware circuitry, which can execute these functions, may be assembled in the control circuitry 23 itself, or may be assembled in the main body device 10 as circuitry to which the control circuitry 23 can refer.

The control circuitry 23 may be realized by an application-specific integrated circuit (ASIC) in which this dedicated hardware circuitry is assembled, a field programmable logic device (FPGA), a complex programmable logic device (CPLD), or a simple programmable logic device (SPLD).

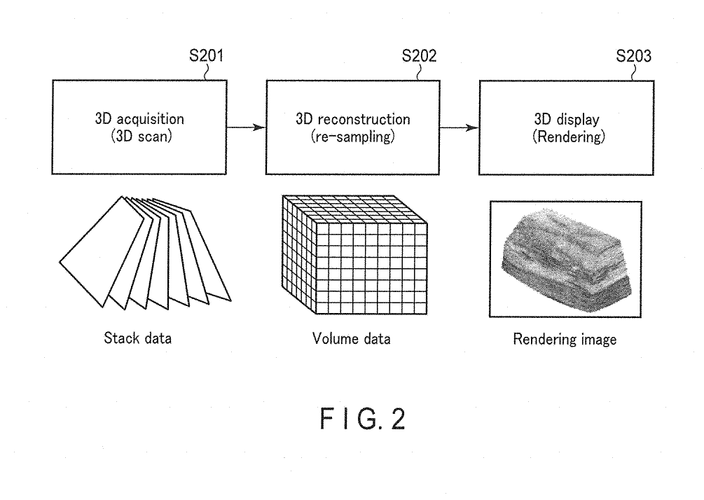

Next, referring to FIG. 2, a description will be given of three-dimensional display (3D display) and four-dimensional display (4D display) of ultrasonic image data acquired by the ultrasonic diagnostic apparatus 1. A process illustrated in FIG. 2 may be executed by the three-dimensional processing circuitry 15, or may be executed by the control circuitry 23.

An upper part of FIG. 2 illustrates, by steps, a flow from acquisition to display of ultrasonic data. A lower part of FIG. 2 illustrates the state of data obtained by each step.

In step S201, for example, the user three-dimensionally scans the ultrasonic probe 70. Thereby, three-dimensional image data is acquired as stack data. A three-dimensional repetitive scan is enabled by an electronic scan in which a mechanical 4D probe or a two-dimensional array probe is used as the ultrasonic probe 70. Thus, it is possible to acquire ultrasonic image data of four dimensions including a time axis, this ultrasonic image data being three-dimensional image data which are temporally successively acquired.

In step S202, since a plurality of two-dimensional ultrasonic image data (tomographic images), which are the acquired stack data, are acquired at mutually different coordinates, a coordinate system which can be commonly used between the respective tomographic images, is introduced. Thus, the three-dimensional ultrasonic image data are reconstructed (re-sampled) as isotropic voxels, and volume data is obtained.

In step S203, the volume data is project-displayed (rendered) by projection from the three dimensions onto a two-dimensional plane. Examples of the rendering method include an MPR (Multi-Planar Reconstruction/Reformation) method, an MIP (Maximum Intensity Projection) method, and a VR (Volume Rendering) method.

The MPR method is a method of creating a tomographic image in an arbitrary direction. A pixel value is calculated by interpolating a voxel value near a designated tomographic plane. The MPR method is useful in that a cross section, which cannot be viewed by normal ultrasonic imaging, can be observed. Normally, in order to grasp a stereoscopic structure, three cross sections, which are a combination of a designated cross section and two cross sections perpendicular to the designated cross section, are displayed at the same time.

The MIP method is a display method in which voxel values existing on a straight line between a point of view and a projection surface are checked, and the maximum value of the voxel values is projected on the projection plane. This method is useful, for example, in stereoscopic depiction of a blood vessel image by a color Doppler method or a contrast echo image in an ultrasonic contrast echo method. However, since depth information disappears in the MIP method, images created at varied angles need to be rotated and cine-displayed.

The VR method is a method in which a virtual physical phenomenon is simulated. In the virtual physical phenomenon, uniform light is emitted from a virtual screen, and the emitted light is reflected, attenuated and absorbed by a three-dimensional object which is expressed by voxel values. Transmissive light and reflective light are updated at intervals of a fixed step from a point on the virtual screen, which is the start point. At a time of the update process, an opacity corresponding to a voxel value is set. Thereby, various expressions can be realized in a range from a surface to an internal structure of the living body. In particular, this method is excellent in extracting a fine structure.

(Alignment Between Ultrasonic Image Data)

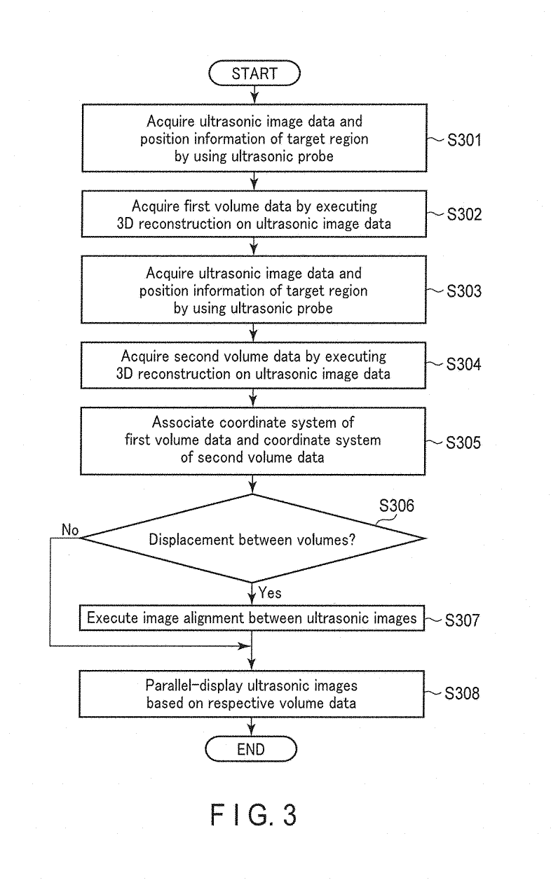

Referring to a flowchart of FIG. 3, a first embodiment will be described. The first embodiment relates to an alignment process between ultrasonic image data and medical image data. The medical image data is ultrasonic image data, and the alignment process is executed between ultrasonic image data which are acquired at different times. In the present embodiment, for example, a case of a treatment of liver cancer is assumed. In this case, before the treatment, ultrasonic image data of the vicinity of the liver cancer is acquired. After the treatment, ultrasonic image data of the vicinity of the treated liver cancer is acquired once again. The images before and after the treatment are compared, and the effect of the treatment is determined.

In step S301, the ultrasonic probe 70 of the ultrasonic diagnostic apparatus according to the present embodiment is operated. Thereby, the control circuitry 23, which executes the data acquisition function 102, acquires ultrasonic image data of a living body region (also referred to as “target region”) in the vicinity of the liver cancer that is the treatment target. In addition, the control circuitry 23, which executes the position information acquisition function 101, acquires the position information of the ultrasonic probe 70 at the time of acquiring the ultrasonic image data from the position sensor system 30, and generates the ultrasonic image data with position information.

In step S302, the control circuitry 23 or three-dimensional processing circuitry 15 executes three-dimensional reconstruction of the ultrasonic image data by the above-described procedure illustrated in FIG. 2, by using the ultrasonic image data and the position information of the ultrasonic probe 70, and generates the volume data (also referred to as “first volume data”) of the ultrasonic image data with position information. In the meantime, since this ultrasonic image data is ultrasonic image data with position information before the treatment, the ultrasonic image data with position information is stored in the image database 20 as past ultrasonic image data.

Thereafter, a stage is assumed in which the treatment progressed and the operation was finished, and the effect of the treatment is determined.

In step S303, like step S301, the control circuitry 23, which executes the position information acquisition function 101 and the data acquisition function 102, acquires the position information of the ultrasonic probe 70 and ultrasonic image data. Like the operation before the treatment, the ultrasonic probe 70 is operated on the target region after the treatment, and the control circuitry 23 acquires the ultrasonic image data of the target region, acquires the position information of the ultrasonic probe 70 from the position sensor system, and generates the ultrasonic image data with position information.

In step S304, like step S302, the control circuitry 23 or three-dimensional processing circuitry 15 generates volume data (also referred to as “second volume data”) of the ultrasonic image data with position information, by using the acquired ultrasonic image data and position information.

In step S305, based on the acquired position information of the ultrasonic probe 70 and ultrasonic image data, the control circuitry 23, which executes the sensor alignment function 103, executes sensor alignment between the coordinate system (also referred to as “first coordinate system”) of the first volume data and the coordinate system (also referred to as “second coordinate system”) of the second volume data, so that the positions of the target regions may generally match. Both the position of the first volume data and the position of the second volume data are commonly described in the position sensor coordinate system. Accordingly, the alignment can directly be executed based on the position information added to each volume data.

In step S306, if the living body does not move during the period from the acquisition of the first volume data to the acquisition of the second volume data, a good alignment state can be obtained by only the sensor alignment. In this case, parallel display of ultrasonic images in step S308 in FIG. 3 is executed. If a displacement occurs in the sensor coordinate system due to a motion of the body or the like, image alignment of step S307 is executed. If the alignment result is good, parallel display of ultrasonic images in step S308 is executed.

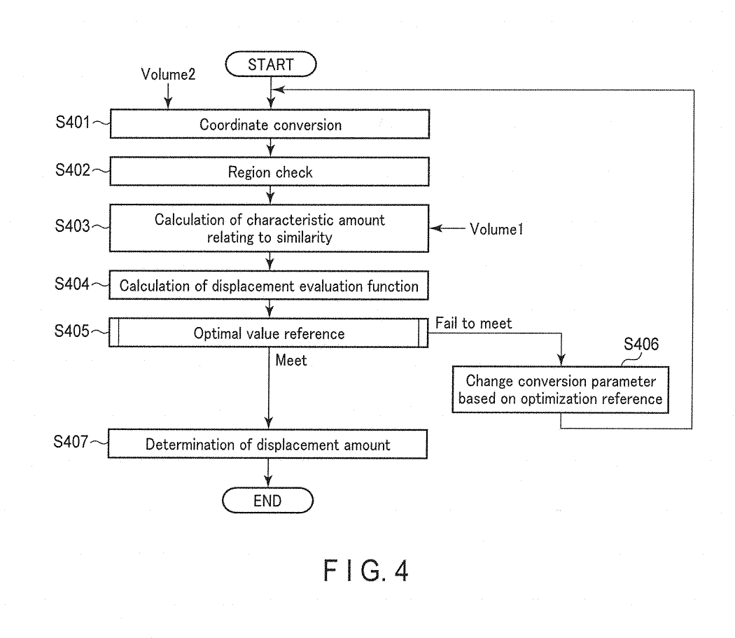

The details of the image alignment will be described later with reference to FIG. 4.

In step S308, the control circuitry 23 instructs, for example, the display processing circuitry 17 to parallel-display the ultrasonic image before the treatment, which is based on the first volume data, and the ultrasonic image after the treatment, which is based on the second volume data. By the above, the alignment process between ultrasonic image data is completed.

Next, referring to a flowchart of FIG. 4, a description will be given of an image alignment process by the control circuitry 23, which the image alignment function illustrated in step S307 realizes.

In step S401, the control circuitry 23 converts the coordinates with respect to one of the first volume data and the second volume data, to be more specific, the second volume data in this example. The coordinate conversion may be executed based on at least six parameters, namely the rotational movements and translational movements in an X direction, Y direction and Z direction, and, if necessary, based on nine parameters which additionally include three shearing directions.

In step S402, the control circuitry 23 checks a coordinate-converted region. Specifically, for example, the control circuitry 23 excludes data other than the volume data region. The control circuitry 23 may generate, at the same time, an arrangement in which an inside of the region is expressed by “1” and an outside of the region is expressed by “0”. In addition, the control circuitry 23 may set a specific pixel value (e.g. 255) for the outside of the region, and may represent the brightness by 0 to 254.

In step S403, the control circuitry 23 calculates a characteristic amount relating to the similarity between the first volume data and the second volume data. The characteristic amount is, for example, a brightness value of a voxel.

In step S404, the control circuitry 23 calculates an evaluation function of displacement between the first volume data and second volume data. As the evaluation function, for example, use may be made of a mutual information amount such as a brightness difference between brightness values calculated in step S403, a correlation, or a region with a highest similarity searched after matching structural information of brightness between volume data.

In step S405, the control circuitry 23 determines whether or not the evaluation function meets an optimal value reference. If the evaluation function meets the optimal value reference, the process advances to step S406. If the evaluation function fails to meet the optimal value reference, the process advances to step S406. Whether or not to meet the optimal value reference may be determined such that the evaluation function is determined to meet the optimal value reference at a time point when an improvement of the reference of similarity is no longer desired.

In step S406, the control circuitry 23 changes the conversion parameter in accordance with the result of the optimal value reference. When the improvement of the reference of similarity is no longer desired, it is possible that the similarity reference falls in a local solution. As a matter of course, the similarity reference at this time is less than the similarity reference of the optimal solution, and can be determined by comparing the ratio to the similarity reference of the image at a time of a large displacement, with the similarity reference at a time of an empirically recognized optimal solution. If it is determined that the similarity reference falls in the local solution, the parameter is slightly changed from the position at that time, and the optimization is executed once again. Thereby, it can be expected that the similarity reference reaches the optimal solution. For example, in the case of a downhill simplex method, the change of the parameter is implemented by making an initially set simplex position greater than the previous one.

In step S407, the control circuitry 23 determines a displacement amount, and makes a correction by the displacement amount. Thus, the image alignment process is completed. The image alignment illustrated in FIG. 4 is merely an example, and general methods relating to the image alignment may be used.

FIG. 5 illustrates an example of the alignment between 3D ultrasonic image data which was described with reference to FIG. 3.

A left image in FIG. 5 is an ultrasonic image before a treatment, which is based on the first volume data. A right image in FIG. 5 is an ultrasonic image after the treatment, which is based on the second volume data. The state of FIG. 5 shows the state of step S305 of FIG. 3. In the description below, the ultrasonic image is illustrated by black-and-white reverse display. As illustrated in FIG. 5, if the time of acquisition of ultrasonic image data differs, a displacement may occur due to a body motion or the like, even if the same target region is scanned.

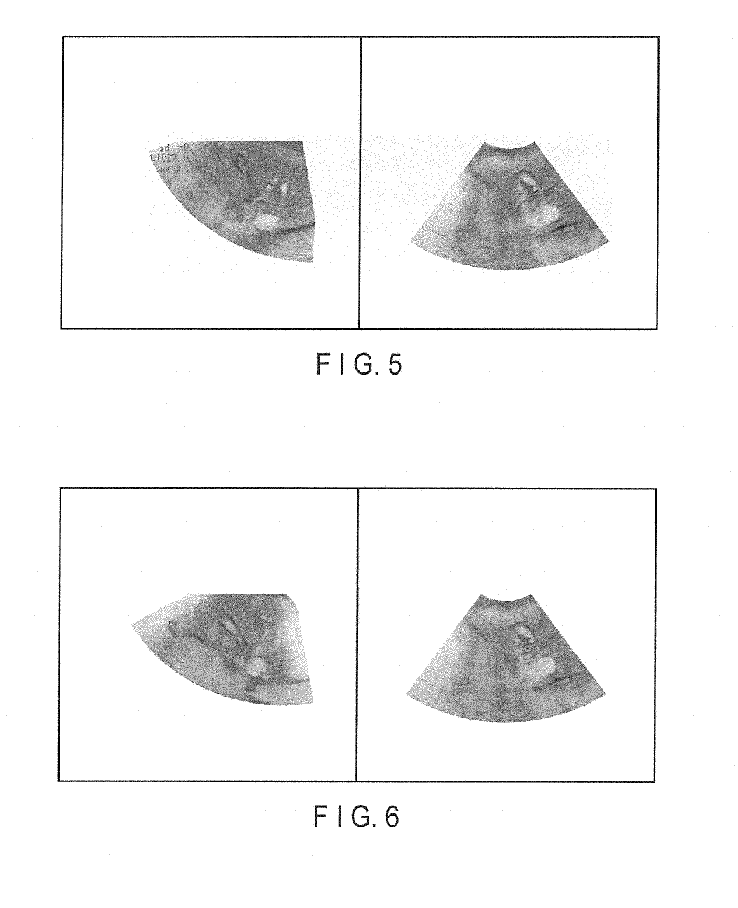

Next, referring to FIG. 6, a description will be given of an example of the ultrasonic image display after the image alignment illustrated in step S308.

A left image in FIG. 6 is an ultrasonic image based on the first volume data before a treatment. A right image in FIG. 6 is an ultrasonic image based on the second volume data after the treatment. As illustrated in FIG. 6, the ultrasonic image data before and after the treatment are aligned, and the ultrasonic image based on the first volume data is rotated in accordance with the position of the ultrasonic image based on the second volume data, and both images are displayed in parallel. As illustrated in FIG. 6, since the alignment between the ultrasonic images is completed, the user can search and display a desired cross section in the aligned state, for example, by a panel operation, and can easily understand the evaluation of the target region (the treatment state of the treatment region).

(Correction of Displacement Due to Body Motion or Respiratory Time Phase).

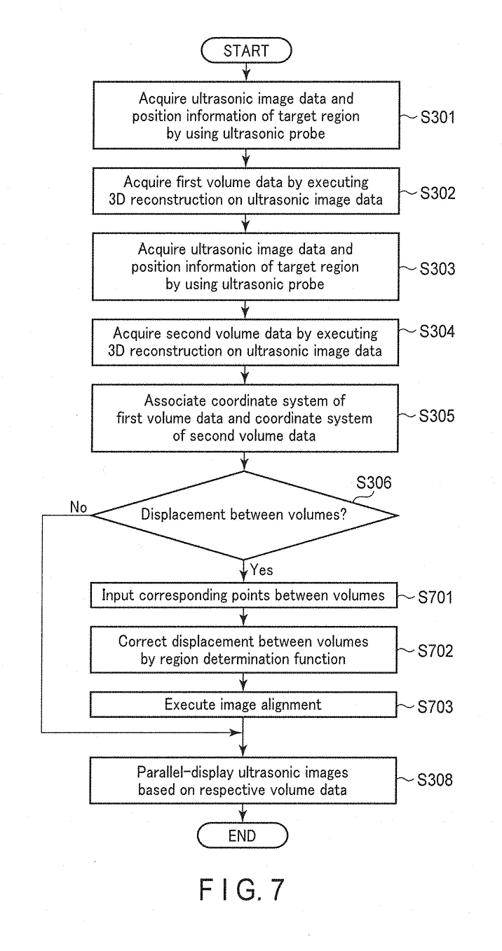

A second embodiment will be described with reference to FIG. 7.

During a treatment, in some cases, due to a body motion, a large displacement t occurs between ultrasonic image data in the position sensor coordinate system, and this displacement exceeds a correctable range of image alignment. There is also a case in which a transmitter of a magnetic field is moved to a position near the patient, from the standpoint of maintaining the magnetic field strength. In such cases, even after the coordinate system of the sensor is associated by the sensor alignment function 103, a case is assumed in which a large displacement remains between the ultrasonic image data. In connection with such a case, a flowchart of FIG. 7 is illustrated as the second embodiment. If it is judged in step S306 that a large displacement remains after the sensor alignment, a process of step S701 is executed.

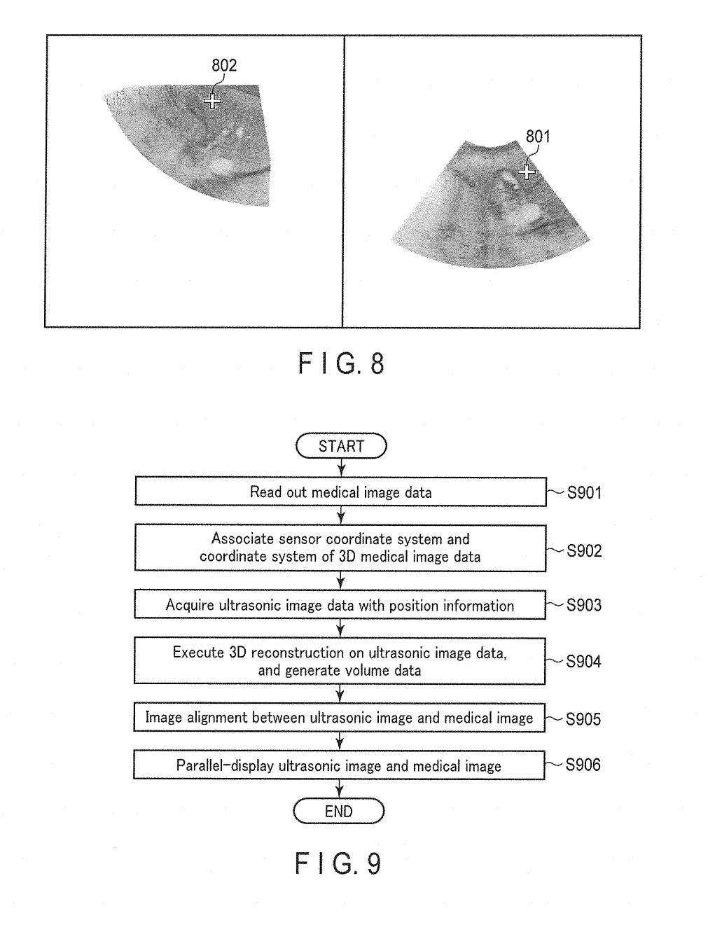

The user designates, in the respective ultrasonic images, corresponding points indicative of a living body region, these points corresponding between the ultrasonic image based on the first volume data and the ultrasonic image based on the second volume data. The method of designating the corresponding points may be, for example, a method in which the user designates the corresponding points by moving a cursor on the screen by using the operation panel through the user interface generated by the display processing circuitry 17, or the user may directly touch the corresponding points on the screen in the case of a touch screen. In an example of FIG. 8, the user designates a corresponding point 801 on the ultrasonic image based on the first volume data, and designates a corresponding point 802, which corresponds to the corresponding point 801, on the ultrasonic image based on the second volume data. The control circuitry 23 displays the designated corresponding points 801 and 802, for example, by “+” marks. Thereby, the user can easily understand the corresponding points, and the user can be supported in inputting the corresponding points. The control circuitry 23, which executes the region determination function 104, calculates a displacement between the designated corresponding points 801 and 802, and corrects the displacement. The displacement may be corrected, for example, by calculating, as a displacement amount, a relative distance between the corresponding point 801 and corresponding point 802, and by moving and rotating, by the displacement amount, the ultrasonic image based on the second volume data.

In the meantime, a region of a predetermined range in the corresponding living body region may be determined as the corresponding region. Also in the case of designating the corresponding region, the control circuitry 23 may execute a similar process as in the case of the corresponding points.

Furthermore, although the example of correcting the displacement due to the body motion or respiratory time phase has been illustrated, the corresponding points or corresponding regions may be determined in order for the user to designate a region-of-interest (ROI) in the image alignment.

Like the first embodiment, after the displacement between the ultrasonic images was corrected by step S702 of FIG. 7, an instruction for image alignment is input, for example, by the user operating the operation panel or pressing the button attached to the ultrasonic probe 70. The image alignment function of step S703 of FIG. 7 may execute image alignment, based on the ultrasonic image data in which displacement was corrected. Like the flowchart of FIG. 3, a transition occurs to the state of FIG. 6.

After the input of the instruction for image alignment, the display processing circuitry 17 parallel-displays the ultrasonic images which are aligned in step S308 of FIG. 7. Thereby, the user can observe the images by freely varying the positions and directions of the images, for example, by the operation panel of the ultrasonic diagnostic apparatus 1. In the 3D ultrasonic image data, the positional relationship between the first volume data and second volume data is interlocked, and MPR cross sections can be moved and rotated in synchronism. Where necessary, the synchronization of MPR cross sections can be released, and the MPR cross sections can independently be observed. In place of the operation panel of the ultrasonic diagnostic apparatus 1, the ultrasonic probe 70 can be used as the user interface for moving and rotating the MPR cross sections. The ultrasonic probe 70 is equipped with a magnetic sensor, and the ultrasonic diagnostic apparatus 1 can detect the movement amount, rotation amount and direction of the ultrasonic probe 70. By the movement of the ultrasonic probe 70, the positions of the first volume data and second volume data of the 3D ultrasonic image data can be synchronized, and the first volume data and second volume data can be moved and rotated.

(Alignment Between Ultrasonic Image Data and Medical Image Data Other than Ultrasonic Image)

A third embodiment will be described.

Hereinafter, a description will be given of a case of executing alignment between medical image data which is obtained by other modalities, such as CT image data, MR image data, X-ray image data and PET image data, and ultrasonic image data which is currently acquired by using the ultrasonic probe 70. In the description below, the case is assumed in which MRI image data is used as the medical image data.

Referring to a flowchart of FIG. 9, an alignment process between the ultrasonic image data and the medical image data will be described. Although three-dimensional image data is assumed as the medical image data, four-dimensional image data may be used as the medical image data, as needed.

In step S901, the control circuitry 23 reads out 3D medical image data from the image database 20.

In step S902, the control circuitry 23 executes associating between the sensor coordinate system of the position sensor system 30 and the coordinate system of the 3D medical image data.

In step S903, the control circuitry 23, which executes the position information acquisition function 101 and the data acquisition function 102, associates the ultrasonic image data, which is acquired by the ultrasonic probe 70, and the position information at a time when the ultrasonic image data is acquired, thereby acquiring ultrasonic image data with position information.

In step S904, the control circuitry 23 or three-dimensional processing circuitry 15 generates volume data of the ultrasonic image data with position information.

In step S905, like step S307, the control circuitry 23, which executes the image alignment function 105, executes alignment between the volume data and the 3D medical image data.

In step S906, the display processing circuitry 17 parallel-displays the ultrasonic image based on the volume data and the medical image based on the 3D medical image data.

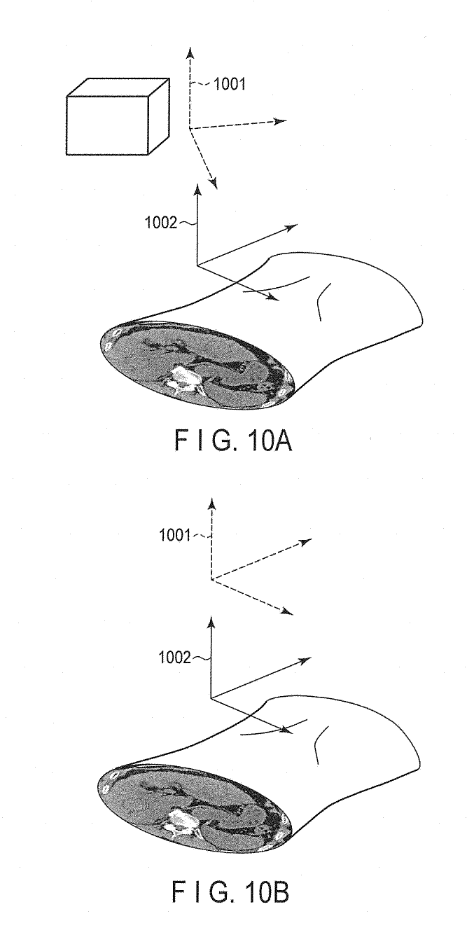

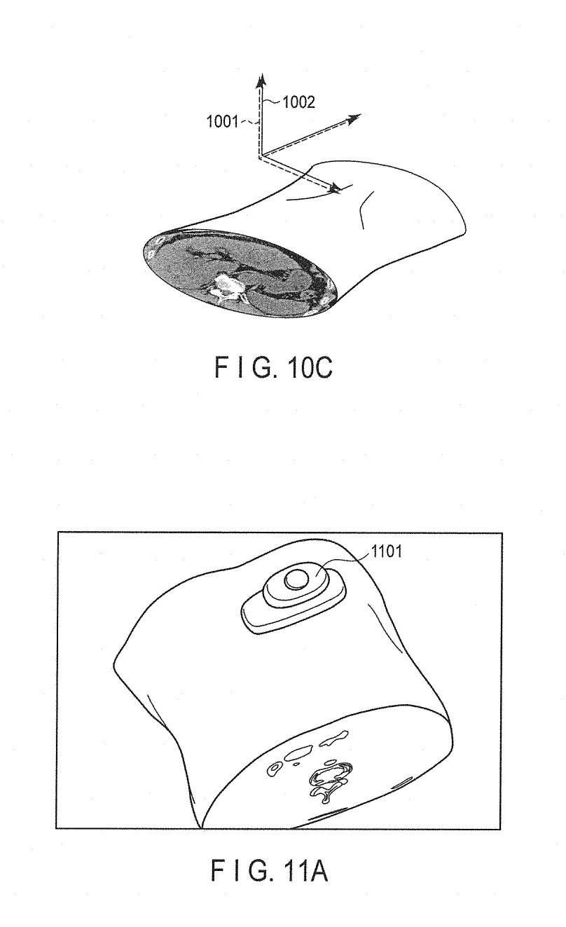

Next, referring to FIG. 10A, FIG. 10B and FIG. 10C, a description will be given of the associating between the sensor coordinate system and the coordinate system of the 3D medical image data, which is illustrated in step S902. This associating is a sensor alignment process corresponding to step S306 of the flowchart of FIG. 3.

FIG. 10A illustrates an initial state. As illustrated in FIG. 10A, a position sensor coordinate system 1001 of the position sensor system for generating the position information which is added to the ultrasonic image data, and a medical image coordinate system 1002 of medical image data, are independently defined.

FIG. 10B illustrates a process of alignment between the respective coordinate systems. The coordinate axes of the position sensor coordinate system 1001 and the coordinate axes of the medical image coordinate system 1002 are aligned in identical directions. Specifically, the directions of the coordinate axes of the coordinate systems are uniformized.

FIG. 10C illustrates a process of mark alignment. FIG. 10C illustrates a case in which the coordinates of the position sensor coordinate system 1001 and the coordinates of the medical image coordinate system 1002 are aligned in accordance with a predetermined reference point. Between the coordinate systems, not only the directions of the axes, but also the positions of the coordinates can be made to match.

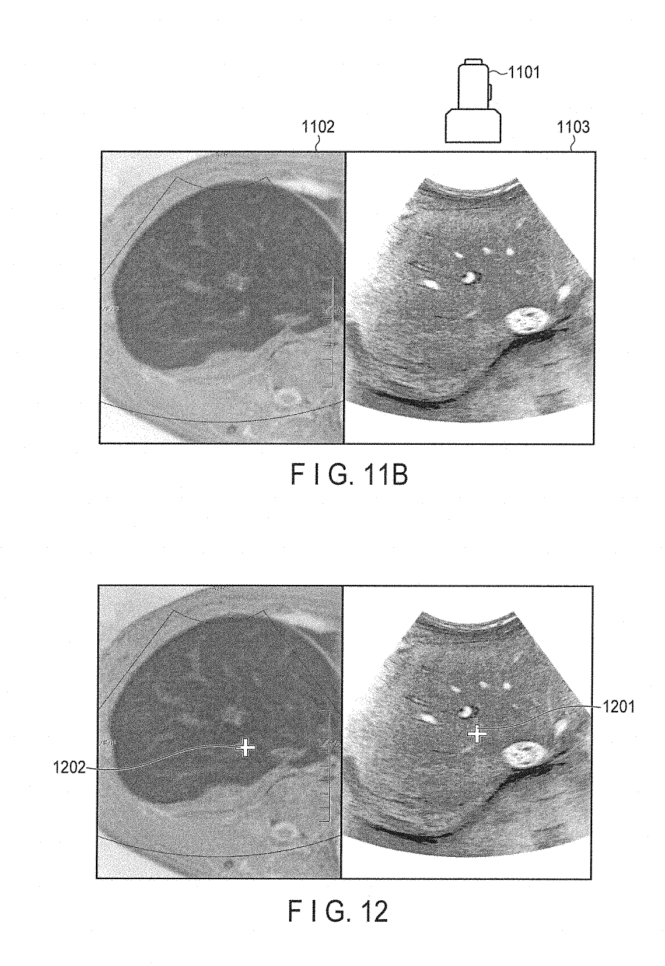

Referring to FIG. 11A and FIG. 11B, a description will be given of a process of realizing, in an actual apparatus, the associating between the sensor coordinate system and the coordinate system of the 3D medical image data.

FIG. 11A is a schematic view illustrating an example of the case in which a doctor performs an examination of the liver. The doctor places the ultrasonic probe 70 horizontally on the abdominal region of the patient. In order to obtain an ultrasonic tomographic image in the same direction as an axial image of CT or MR, the ultrasonic probe 70 is disposed in a direction perpendicular to the body axis, and in such a direction that the ultrasonic tomographic image becomes vertical from the abdominal side toward the back. Thereby, an image as illustrated in FIG. 11B is acquired. In the present embodiment, in step S901, a three-dimensional MR image is read in from the image database 20, and this three-dimensional MR image is displayed on the left side of the monitor. The MR image of the axial cross section, which is acquired at the position of an icon 1101, is an MR image 1102 illustrated in FIG. 11B, and is displayed on the left side of the monitor. Furthermore, a real-time ultrasonic image 1103, which is updated in real time at that time, is displayed on the right side of the monitor in parallel with the MR image 1102. By disposing the ultrasonic probe 70 on the abdominal region as illustrated in FIG. 11A, the ultrasonic tomographic image in the same direction as the axial plane of the MR can be acquired.

The user puts the ultrasonic probe 70 on the body surface of the living body in the direction of the axial cross section. The user confirms, by visual observation, whether or not the ultrasonic probe 70 is in the direction of the axial cross section. When the user puts the ultrasonic probe 70 on the living body in the direction of the axial cross section, the user performs a registration process such as clicking by the operation panel, or pressing of the button. Thereby, the control circuitry 23 acquires and associates the sensor coordinates of the position information of the sensor of the ultrasonic probe 70 in this state, and the MR data coordinates of the position of the MPR plane of the MR data. The axial cross section in the MR image data of the living body can be converted to the position sensor coordinates, and can be recognized. Thereby, the alignment (matching of directions of coordinate axes of coordinate systems) illustrated in FIG. 11B is completed. In the alignment state, the system can associate the MPR image of the MR and the real-time ultrasonic tomographic image by the sensor coordinates, and can display these images in interlocking manner. At this time, since the axes of both coordinate systems are coincident, the directions of the images match, but a displacement remains in the position of the body axis direction. By moving the ultrasonic probe 70 in the state in which the displacement remains in the position of the body axis direction, the user can observe the MPR plane of the MR and the real-time ultrasonic image in an interlocking manner.

Next, referring to FIG. 12, a description will be given of the method of realizing, by the apparatus, the process of the mark alignment illustrated in FIG. 10C.

FIG. 12 illustrates a parallel-display screen of the MR image 1102 and real-time ultrasonic image 1103 illustrated in FIG. 11B, the parallel-display screen being displayed on the monitor.

After the completion of the alignment, by moving the ultrasonic probe 70 in the state in which the displacement remains in the position of the body axis direction, the user can observe the MPR plane of the MR and the real-time ultrasonic image in an interlocking manner.

While viewing the real-time ultrasonic image 1103 which is displayed on the monitor, the user scans the ultrasonic probe 70, thereby causing the monitor to display a target region (or an ROI) such as the center of the region for alignment or a structure. Thereafter, the user designates the target region as a corresponding point 1201 by the operation panel or the like. In the example of FIG. 12, the designated corresponding point is indicated by “+”. At this time, the system acquires and stores the position information of the sensor coordinate system of the corresponding point 1201.

Next, the user moves the MPR cross section of the MR by moving the ultrasonic probe 70, and displays the cross-sectional image of the MR image, which corresponds to the cross section including the corresponding point 1201 of the ultrasonic image designated by the user. When the cross-sectional image of the MR image, which corresponds to the cross section including the corresponding point 1201, was displayed, the user designates a target region (or an ROI), such as the center of the region for alignment or a structure, which is designated on the cross-sectional image of the MR image, as a corresponding point 1202 by the operation panel or the like. At this time, the system acquires and stores the position information of the coordinate system of the MR data of the corresponding point 1202.

The control circuitry 23, which executes the region determination function, corrects a displacement between the coordinate system of the MR image data and the sensor coordinate system, based on the position of the designated corresponding point in the sensor coordinate system and the position of the designated corresponding point in the coordinate system of the MR data. Specifically, for example, based on a difference between the corresponding point 1201 and corresponding point 1202, the control circuitry 23 corrects a displacement between the coordinate system of the MR image data and the sensor coordinate system, and aligns the coordinate systems. Thereby, the process of mark alignment of FIG. 10C is completed, and the step S902 of the flowchart of FIG. 9 is finished.

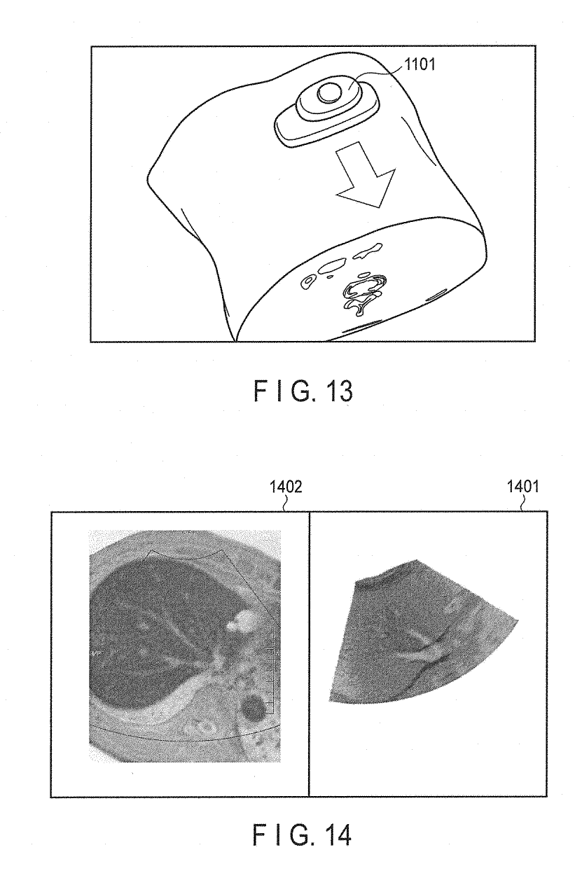

Next, referring to a schematic view of FIG. 13, a description will be given of an example of acquisition of ultrasonic image data in the step S903 of the flowchart of FIG. 9, in the state in which the coordinate system of the MR data and the sensor coordinate system are aligned.

After the completion of the position correction, the user manually operates the ultrasonic probe 70 with respect to the region including the target region, while referring to the three-dimensional MR image data, and acquires the ultrasonic image data with position information. FIG. 13 is a schematic view illustrating that the user manually moves the ultrasonic probe 70 on the abdominal region.

Next, the user presses the switch for image alignment, and executes image alignment. By the process thus far, the position of the MR data and the position of the ultrasonic data are made to generally match, and the MR data and the ultrasonic data include the common target. Thus, the operation of image alignment is well performed. An example of the ultrasonic image display after the image alignment will be described with reference to FIG. 14. As in the step S906 of FIG. 9, the ultrasonic image, which is aligned with the MR image, is parallel-displayed.

As illustrated in FIG. 14, an ultrasonic image 1401 of ultrasonic image data is rotated and displayed in accordance with the image alignment, so as to correspond to an MR 3D image 1402 of MR 3D image data. Thus, it becomes easier to understand the positional relationship between the ultrasonic image and MR 3D image. It is possible to observe the image by freely changing the position and direction of the image by the operation panel or the like of the ultrasonic diagnostic apparatus 1. The positional relationship between the MR 3D image data and the 3D ultrasonic image data is interlocked, and the MPR cross sections can be synchronously moved and rotated. Where necessary, the synchronization of MPR cross sections can be released, and the MPR cross sections can independently be observed. In place of the operation panel of the ultrasonic diagnostic apparatus 1, the ultrasonic probe 70 can be used as the user interface for moving and rotating the MPR cross sections. The ultrasonic probe 70 is equipped with the magnetic sensor, and the ultrasonic diagnostic apparatus 1 can detect the movement amount, rotation amount and direction of the ultrasonic probe 70. By the movement of the ultrasonic probe 70, the positions of the MR 3D data and the 3D ultrasonic image data can be synchronized, and can be moved and rotated.

In the third embodiment, the MR 3D image data was described by way of example. However, the third embodiment is similarly applicable to other 3D medical image data of CT, X-ray, ultrasonic, PET, etc. The associating between the coordinate system of 3D medical data and the coordinate system of the position sensor was described in the steps of alignment and mark alignment illustrated in FIG. 10A, FIG. 10B and FIG. 10C. However, the alignment between the coordinates is possible by various methods. It is possible to adopt some other method, such as a method of executing alignment by designating three or more points in both coordinate systems. Besides, instead of acquiring the ultrasonic image data with position information after the completion of the correction of displacement, it is possible to acquire the ultrasonic image data with position information before the completion of the correction of displacement, to generate the volume data, to designate the corresponding points between the ultrasonic image based on the volume data of the ultrasonic image data and the medical image based on the 3D medical image data, and to correct the displacement.

(Synchronous Display Between Ultrasonic Image and Medical Image)

A fourth embodiment will be described.

If the above-described sensor alignment and image alignment are completed, the relationship between the coordinate system of the medical image (the MR coordinate system in this example) and the position sensor coordinate system is determined. The display processing circuitry 17 refers to the position information of the real-time (live) ultrasonic image acquired by the user freely moving the ultrasonic probe 70 after the completion of the alignment process, and can thereby display the MPR cross section of the corresponding MR. The corresponding cross sections of the highly precisely aligned MR image and real-time ultrasonic image can be interlock-displayed (also referred to as “synchronous display”). Synchronous display can also be executed between 3D ultrasonic images by the same method. Specifically, a 3D ultrasonic image, which was acquired in the past, and a real-time 3D ultrasonic image can be synchronously displayed. In the step S308 of FIG. 3 and FIG. 7 and the step S906 of FIG. 9, the parallel synchronous display of the 3D medical image and the aligned 3D ultrasonic image was illustrated. However, by utilizing the sensor coordinates, the real-time ultrasonic tomographic image can be switched and displayed.

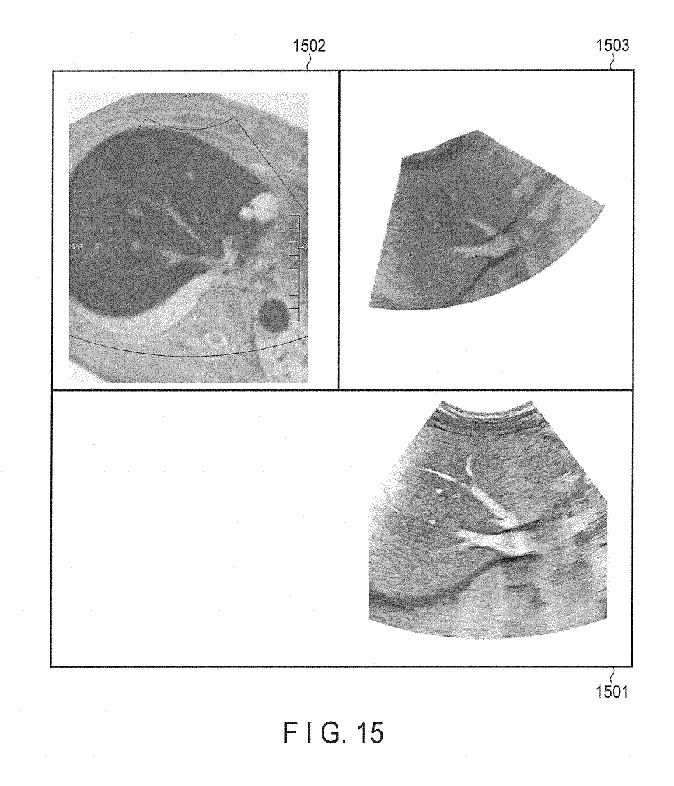

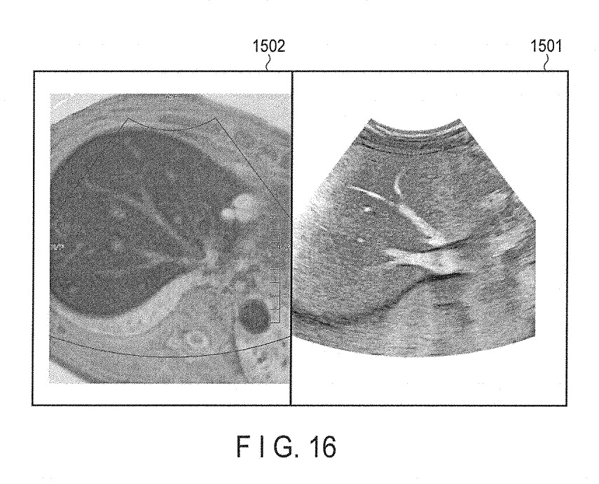

FIG. 15 illustrates an example of synchronous display of the ultrasonic image and medical image by the display processing circuitry 17. For example, if the ultrasonic probe 70 is scanned, a real-time ultrasonic image 1501, a corresponding MR 3D image 1502, and an ultrasonic image 1503 for alignment, which was used for alignment, are displayed. In the meantime, as illustrated in FIG. 16, the real-time ultrasonic image 1501 and MR 3D image 1502 may be parallel-displayed, without displaying the ultrasonic image 1503 for alignment.

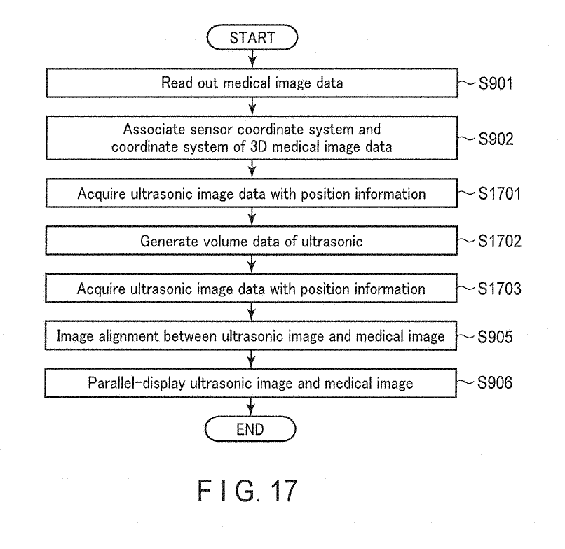

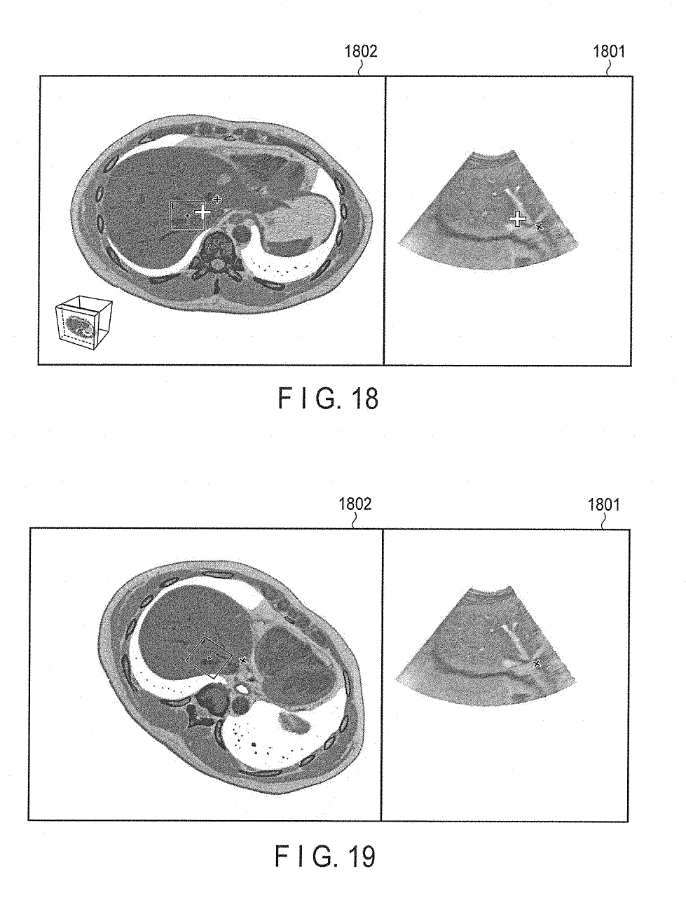

A fifth embodiment will be described. As illustrated in a flowchart of FIG. 17, after the acquisition of the 3D ultrasonic image data, the sensor coordinates and the data coordinates of the 3D medical image data may be associated. For example, in step S1701 and step S1702 in the flowchart of FIG. 17, the control circuitry 23, which executes the data acquisition function 102, reads in the 3D ultrasonic image data, and displays a 3D ultrasonic image 1801 on the right side of the monitor, as illustrated in FIG. 18. The control circuitry 23 reads in a 3D medical image 1802 of 3D medical image data (CT 3D image data in this example) from the image database, and displays the 3D medical image 1802 on the left side of the monitor.

In step S1703 in the flowchart of FIG. 17, the control circuitry 23, which executes the region determination function, determines region information, which is corresponding points or corresponding regions in this example, with respect to the cross section of the CT 3D image and the cross section of the ultrasonic image, as illustrated in FIG. 18. In FIG. 18, the determined positions are displayed by mark “+”. Instead of the corresponding points or corresponding regions, a region at a time of executing a calculation for image alignment can be determined.

The control circuitry 23, which executes the region determination function 104, executes sensor alignment by associating the coordinates of the corresponding point in the data coordinates of the MR, and the coordinates of the corresponding point in the position sensor coordinates.

The control circuitry 23, which executes the image alignment function 105, executes image alignment between the ultrasonic image and medical image, based on the region information. In the state in which the sensor alignment was executed, the user instructs image alignment, for example, by the operation panel. Based on the corresponding region, the control circuitry 23 reads in the CT 3D image data and 3D ultrasonic image data, and executes a process by an image alignment algorithm.

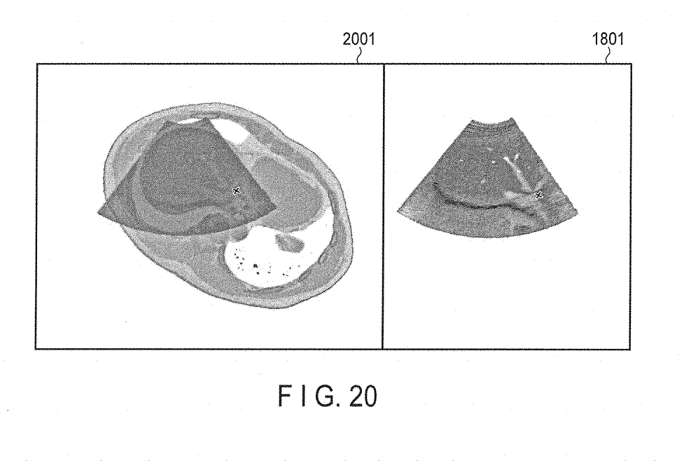

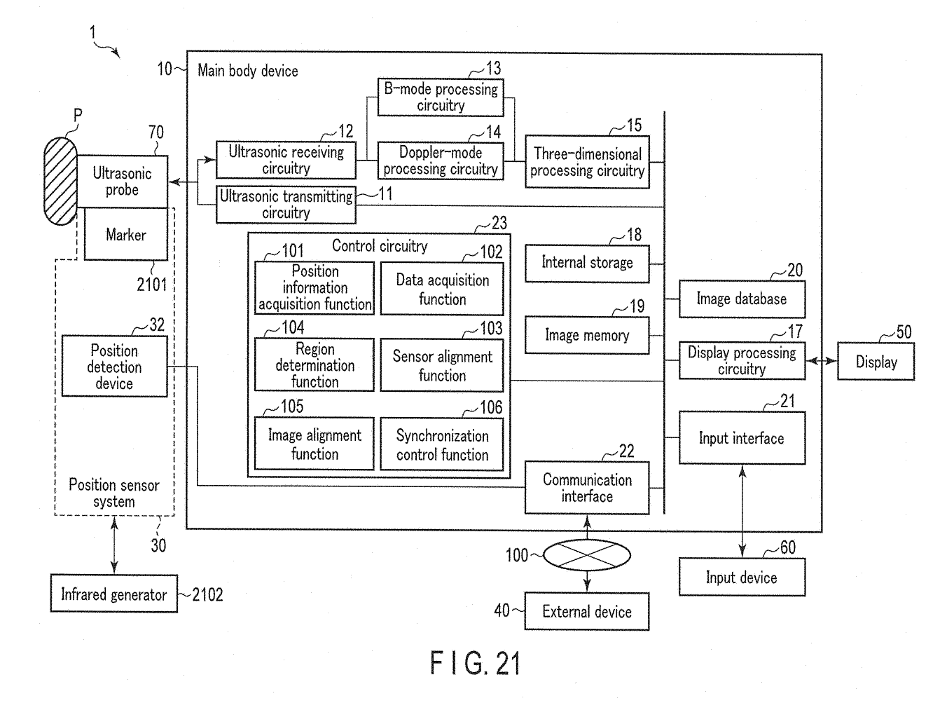

FIG. 19 illustrates a display example of the images after the image alignment process. As illustrated in FIG. 19, the 3D medical image 1802 is rotated and displayed in accordance with the position of the 3D ultrasonic image 1801. In addition, FIG. 20 illustrates a display example of the images after the image alignment process. A corresponding cross section between the CT 3D image and 3D ultrasonic image is displayed as an overlapped display 2001.

According to the above-described embodiment, the coordinate systems between the medical images including ultrasonic image data, which are different with respect to the time of acquisition and the position of acquisition, are associated based on the ultrasonic image data acquired by scanning the ultrasonic probe 70 to which the position information is added by the position sensor system, and the image alignment is executed based on the associating. Thereby, the success rate of image alignment is increased, and the ultrasonic image and medical image, which were easily and exactly aligned, can be presented to the user. In addition, since the sensor coordinate system and the coordinate system of the medical image, for which the image alignment is completed, are synchronized, the MPR cross section of the 3D medical image and real-time ultrasonic tomographic image can be synchronously displayed in interlock with the scan of the ultrasonic probe 70. Specifically, the exact comparison between the medical image and ultrasonic image can be realized, and the objectivity of ultrasonic diagnosis can be improved.

In the above-described embodiments, the position sensor systems, which utilize magnetic sensors, have been described.

FIG. 21 illustrates an embodiment in a case in which infrared is utilized in the position sensor system. Infrared is transmitted at least in two directions by an infrared generator 2102. The infrared is reflected by a marker 2101 which is disposed on the ultrasonic probe 70. The infrared generator 2102 receives the reflected infrared, and the data is transmitted to the position sensor system 30. The position sensor system 30 detects the position and direction of the marker from the infrared information observed from plural directions, and transmits the position information to the ultrasonic diagnostic apparatus.

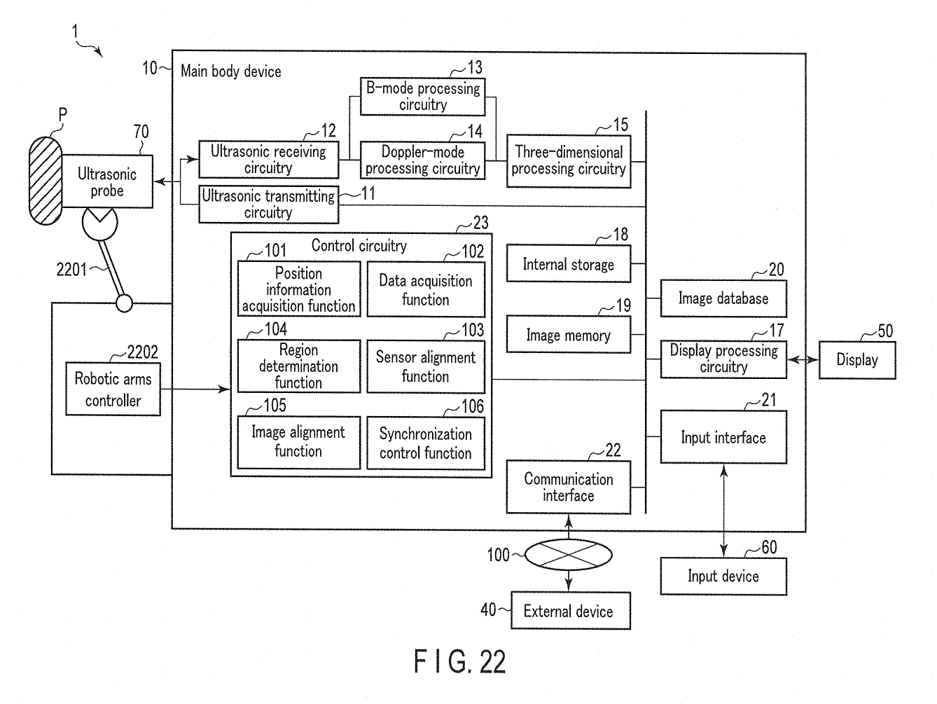

FIG. 22 illustrates an embodiment in a case in which robotic arms are utilized in the position sensor system. Robotic arms 2201 move the ultrasonic probe 70. Alternatively, the doctor moves the ultrasonic probe 70 in the state in which the robotic arms 2201 are attached to the ultrasonic probe 70. A position sensor is attached to the robotic arms 2201, and position information of each part of the robotic arms is successively transmitted to a robotic arms controller 2202. The robotic arms controller 2202 converts the position information to position information of the ultrasonic probe 70, and transmits the converted position information to the ultrasonic diagnostic apparatus.

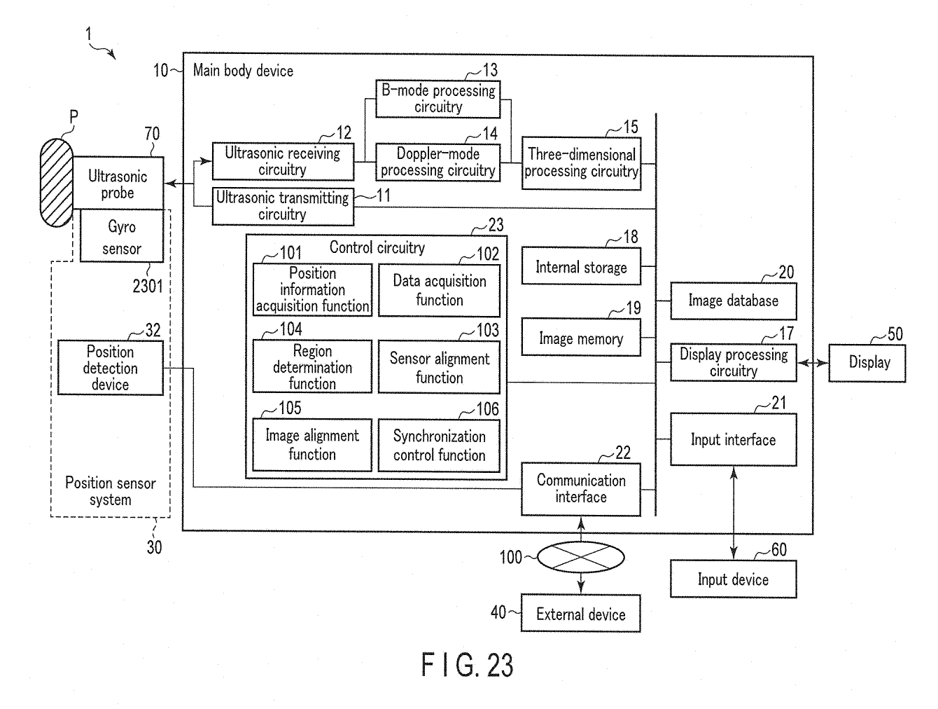

FIG. 23 illustrates an embodiment in a case in which a gyro sensor is utilized in the position sensor system. A gyro sensor 2301 is built in the ultrasonic probe 70, or is disposed on the surface of the ultrasonic probe 70. Position information is transmitted from the gyro sensor 2301 to the position sensor system 30 via a cable. In some cases, as the cable, a part of a cable for the ultrasonic probe 70 may be used, or a dedicated cable may be used. In addition, the position sensor system 30 may be a dedicated unit in some cases, or the position sensor system 30 may be realized by software in the ultrasonic apparatus in other cases. The gyro sensor can integrate an acceleration or rotation information with respect to a predetermined initial position, and can detect changes in position and direction. It can be thought that the position is corrected by GPS information. Alternatively, by an input of the user, initial position setting or correction can be executed. By the position sensor system 30, the information of the gyro sensor is converted to position information by an integration process or the like, and the converted position information is transmitted to the ultrasonic diagnostic apparatus.

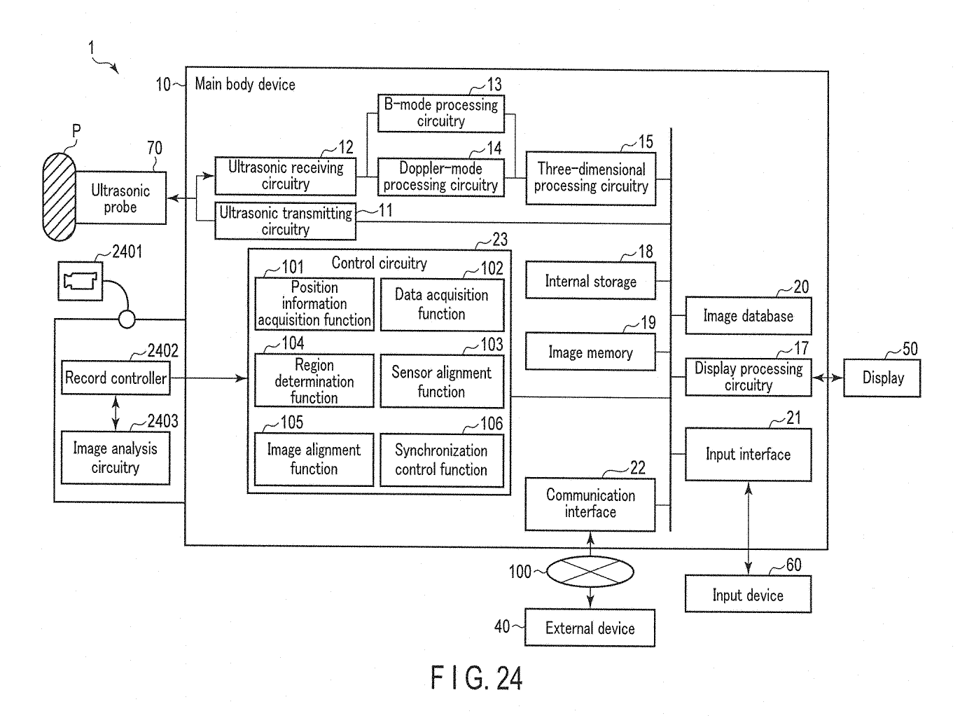

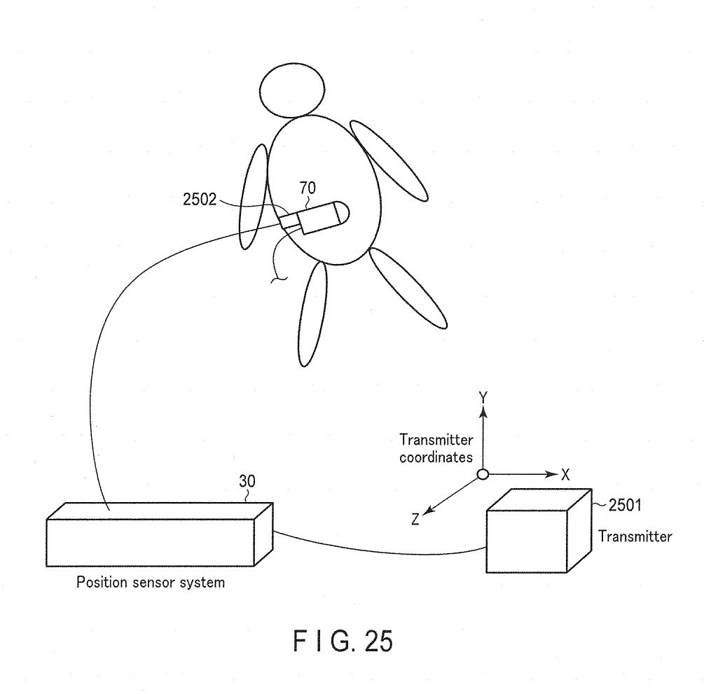

FIG. 24 illustrates an embodiment in a case in which a camera is utilized in the position sensor system. The vicinity of the ultrasonic probe 70 is photographed by a camera 2401 from a plurality of directions. The photographed image is sent to image analysis circuitry 2403, and the ultrasonic probe 70 is automatically recognized and the position is calculated. A record controller 2402 transmits the calculated position to the ultrasonic diagnostic apparatus as position information of the ultrasonic probe 70.

(Modifications of Sensor Alignment Unit)

There are various modifications of the sensor alignment function illustrated in FIG. 1. Although described in the first embodiment to fourth embodiment, such various embodiments will be described once again, and their modifications will be described.

A first embodiment of the sensor alignment unit is as follows. The alignment target region of the 3D medical image data is extracted from the ultrasonic image acquired by the operation of the ultrasonic probe 70. Thus, the sensor alignment unit associates the position sensor coordinates of this ultrasonic image and the coordinates of the corresponding 3D medical image data. This was described in the flowchart of FIG. 9, or in FIG. 12.

A second embodiment of the sensor alignment unit relates to a case in which the 3D medical image data is 3D ultrasonic image data with position information of the position sensor. The flowchart of FIG. 3 illustrates that the sensor alignment unit executes the associating by making use of the common position sensor coordinates. FIG. 25 is a schematic view of a position sensor system by a magnetic sensor. For example, the coordinates of the magnetic field space are defined in a transmitter 2501 of magnetism. By the transmitter coordinates, it is possible to define the position of a magnetic sensor 2502 for ultrasonic probe, which is attached to the ultrasonic probe 70.

When 3D ultrasonic image data is acquired by moving the ultrasonic probe 70, the relationship in position or direction between the 3D ultrasonic image data can be grasped by the common transmitter coordinates, and the alignment can be executed.

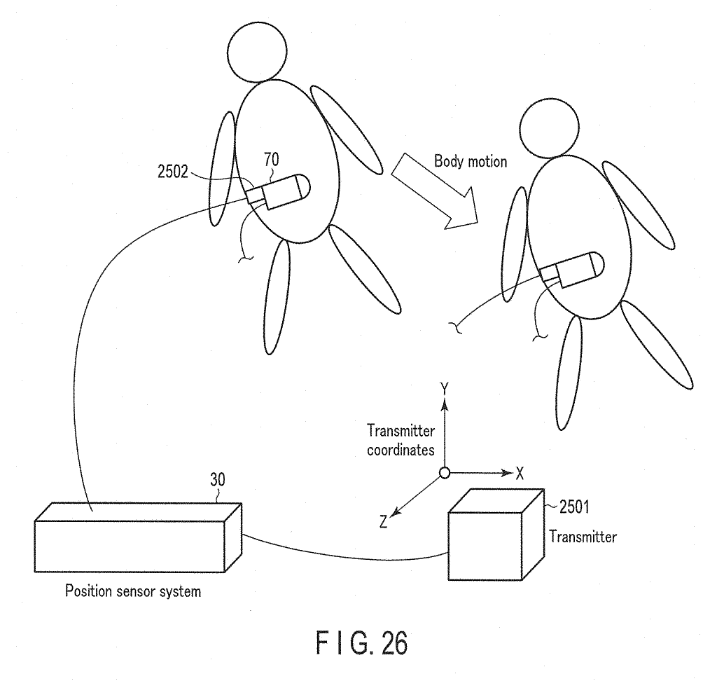

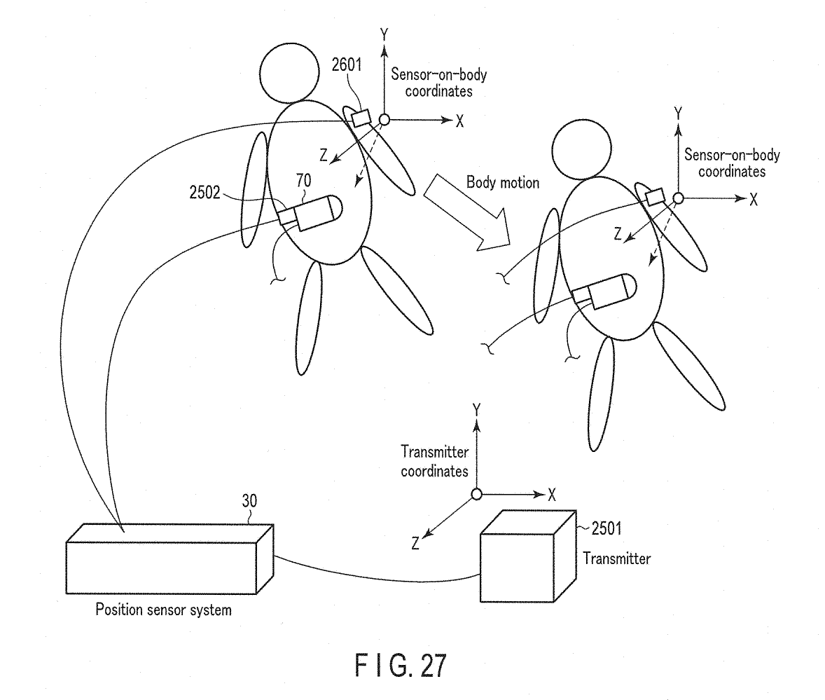

A third embodiment of the sensor alignment unit is a case in which another magnetic sensor is disposed on the body surface. FIG. 26 is a schematic view illustrating a case in which the living body has moved during the ultrasonic examination. The space of the magnetic field is the transmitter coordinate system, and the position of the ultrasonic probe 70 varies due to the movement of the living body. However, there may be a case in which the positional relationship between the living body and the ultrasonic probe 70 is unchanged. In this case, if the 3D ultrasonic image data are aligned by the common transmitter coordinates, as in the second embodiment, a displacement corresponding to the movement of the living body occurs. Thus, as illustrated in FIG. 27, another magnetic sensor 2601 is disposed on the body surface, and a coordinate system of the magnetic field space, which has the origin at the magnetic sensor 2601 on body, is defined. Even if the living body moves as in FIG. 26, the influence of the movement of the living body can be eliminated, as illustrated in FIG. 27, in the body surface sensor coordinates having the origin at the magnetic sensor 2601 on body. As illustrated in FIG. 27, by using the body surface sensor coordinates as the common coordinate system, the relationship in position or direction between the 3D ultrasonic image data is grasped, and the alignment is executed.

The number of robotic arms, which are used as the position sensor system illustrated in FIG. 22, is not limited to one. The position sensor system may include second robotic arms. The second robotic arms are controlled, for example, so as to follow points designated on the body surface of the living body P. The robotic arms controller 2202 controls the movement of the second robotic arms while recognizing the position of the second robotic arms. The control circuitry 23 recognizes that the position, which the second robotic arms follow, is the designated point of the living body. In the meantime, when the designated point exists in the body, the position of the designated point is calculated from the position which the second robotic arms follow, and the position of the determined region in the ultrasonic tomographic image. Thereby, even when the living body P has moved during the examination, or when the body position of the living body P needs to be changed during the examination, the target region of the living body can continuously be recognized.

(Modifications of Ultrasonic Image Data)

In the above, the 3D ultrasonic image data with position information was illustrated as the ultrasonic image data by way of example. However, the ultrasonic image data may be a 2D tomographic image with position information. In the flow of the image alignment process of FIG. 4, for example, Volume 2 can be changed to a 2D tomographic image. By using the 3D ultrasonic image data with position information as Volume 1, the similarity is evaluated while varying the region of the 2D tomographic image of Volume 2, which overlaps the Volume 1. At a stage when the displacement evaluation function meets the reference, the alignment is finished, and the positional relationship between the 3D ultrasonic image data with position information of Volume 1 and the 2D tomographic image of Volume 2 is determined.

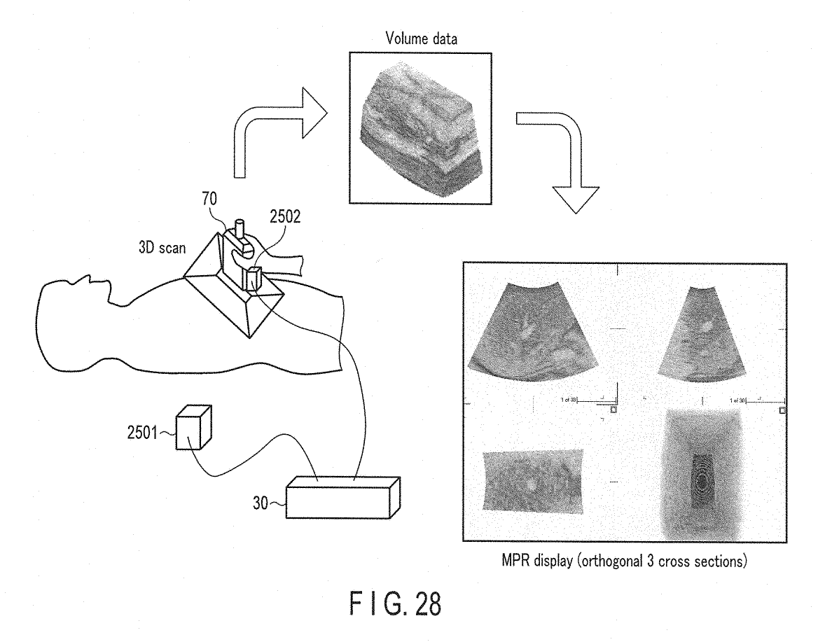

The ultrasonic image data may be 3D ultrasonic image data or 4D ultrasonic image data, which are acquired by electronic scan by a mechanical swing-type 4D probe (mechanical 4D probe) with position information, or a 2D array probe. FIG. 28 illustrates an embodiment in which the position sensor is disposed on the 2D array probe. In the first embodiment, the 3D ultrasonic image data with position information is acquired by manually moving the ultrasonic probe 70. In FIG. 28, the 3D ultrasonic image data can be acquired by electronic control by the 2D array probe. The 3D ultrasonic image data can repetitively be acquired, and position information is added to each 3D ultrasonic image data. The 3D ultrasonic image data used in FIG. 4 or FIG. 9 can be acquired by electronic control by the 2D array probe. By the position information added to the 3D ultrasonic image data, the sensor alignment can be executed in the same manner as in FIG. G. The 2D array probe can continuously generate 3D ultrasonic image data, and can continuously execute the sensor alignment as illustrated in FIG. G. Furthermore, the image alignment can continuously be executed, and the images, which are aligned in real time, can be parallel-displayed on the monitor. The operator can perform diagnosis while varying the observation site by moving the ultrasonic probe 70.

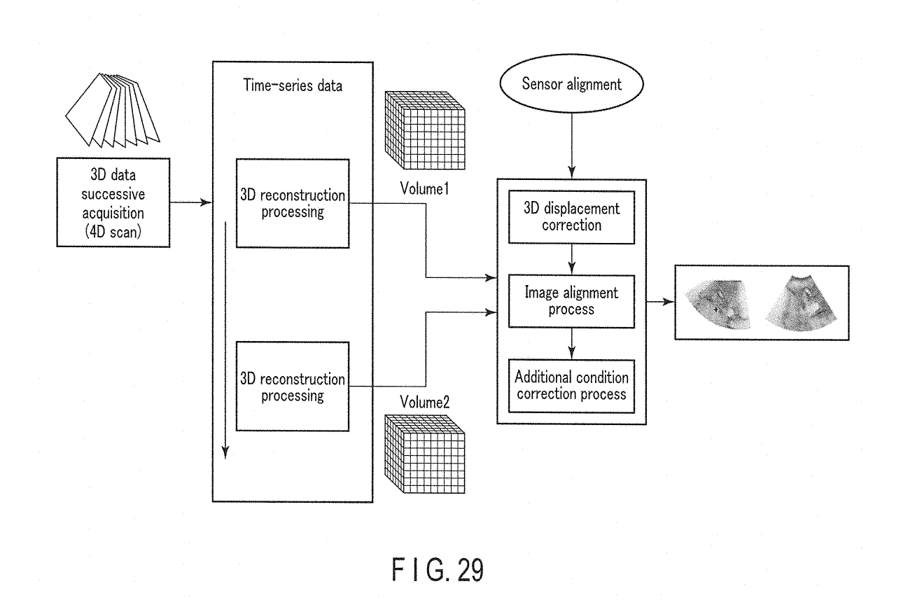

FIG. 29 illustrates a flow of a real-time 3D alignment display process.

As illustrated in FIG. 8, FIG. 12 and FIG. 18, when a displacement occurs due to the movement of the living body or organ, the user designates the alignment center position on the image, thus being able to correct the displacement. In the state in which the displacement is corrected, the image alignment is continuously executed, and the images, which are aligned in real time, can be displayed in parallel on the monitor.

(Modifications of Region Determination Function)

There are various embodiments of the region determination function illustrated in FIG. 1. Although described in the first embodiment to fourth embodiment, such various embodiments will be described once again, and their modifications will be described.

A first embodiment of the region determination function is illustrated in FIG. 7. In the first embodiment, the region determination function is composed of a user interface which determines a corresponding region between the 3D medical image data and 3D ultrasonic image data, and a function of correcting the associating between the position sensor coordinates of the position sensor system and the coordinates of the 3D medical image data, based on the coordinate information of the determined region.

In FIG. 8, if a large displacement remains between the 3D ultrasonic image data, the corresponding region between both 3D ultrasonic images is determined by using the operation panel 4. In FIG. 8, the determined position is displayed by the “+” mark. By using the information of this determination, the region determination function corrects the information of the positional relationship between the 3D ultrasonic image data. By the correction, as in FIG. 6, the state with a displacement within a predetermined range can be realized.

FIG. 18 illustrates an embodiment of the CT 3D image data and 3D ultrasonic image data. The control circuitry 23, which executes the data acquisition function 102, reads in the 3D ultrasonic image data, and the 3D ultrasonic image data is displayed on the right side of the monitor. The CT 3D image data is read in from the image database 20, and the CT 3D image data is displayed on the left side of the monitor. The operator searches, by the operation panel, a cross section including a corresponding region of each data, and the searched cross sections are displayed in parallel. As the corresponding region was determined in the cross section of the MR 3D image of FIG. 12, in the case of FIG. 18, too, the corresponding region between the cross section of the CT 3D image and the ultrasonic cross section is determined. In FIG. 12, the determined position is displayed by the “+” mark. The range of the region, in which the image alignment calculation is performed, can be determined. By using the information of this determination, the region determination function generates the information of the positional relationship between the CT 3D image and the 3D ultrasonic image data.

A second embodiment of the region determination function is illustrated in FIG. 12. In the second embodiment, the region determination function is composed of a user interface which determines a desired target region of 3D medical image data; a user interface which determines a target region of 3D medical image data in a real-time ultrasonic tomographic image by moving the ultrasonic probe 70; a sensor alignment unit including a function of correcting, based on coordinate information of the determined region, the associating between the position sensor coordinates of the position sensor system and the coordinates of the 3D medical image data; and an ultrasonic data acquisition unit which acquires ultrasonic image data in the corrected coordinate relationship.

FIG. 12 illustrates an embodiment of MR 3D image data and 3D ultrasonic image data. As illustrated in FIG. 12, by scanning the ultrasonic probe 70, the center of the region for alignment, or a structure in the region, is determined by the operation panel or the like. Next, by a predetermined user interface, the MR cross section is moved, the MR cross section corresponding to the determined region of the ultrasonic cross section is displayed, and the center of the region for further alignment, or a structure in the region, is determined. In FIG. 12, the determined position is displayed by the “+” mark. The range of the region, in which the image alignment calculation is performed, can also be determined. By using the information of this determination, the region determination function corrects the positional relationship between the MR data coordinates and the position sensor coordinates.

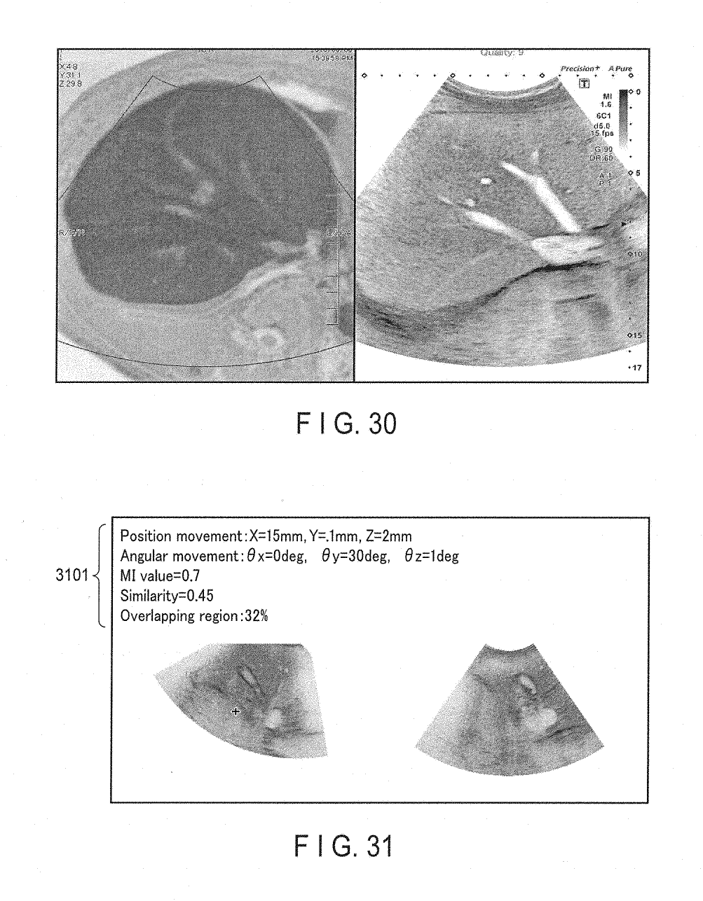

In the region determination function which determines the region information for alignment, image patterns of regions, which are suited for alignment, may be prepared in a database in advance, and 3D medical image data may be automatically searched from the database. FIG. 30 illustrates an example of the liver in an EOB-MRI image and an ultrasonic B-mode image. In the images, hepatic veins are commonly depicted with high quality. When image alignment is executed, the common structure between 3D medical image data is important. In clinical diagnosis, the doctor grasps the relationship between an organ and a tomographic plane, by using a characteristic structure as a clue. Candidates of structures of organs, which the doctor uses as clues for grasping structures, are prepared as a database in advance. As regards the liver, structures of portal veins, hepatic veins, and the surface of the liver are thinkable. As regards the heart, there are typical observation cross sections of four-chamber structures, and there are four-chamber images, two-chamber images, and minor axis images. As regards other organs, there are characteristic structures which the doctor utilizes in grasping structures in diagnosis in advance. An image database of characteristic structures is constructed. The image database is referred to, and the region for alignment is automatically searched by using 3D medical image data which are subjected to alignment. In the example of FIG. 18, the region of, for example, the portal vein is automatically detected from the 3D image data of the MR and ultrasonic, and the candidate cross section is depicted.

In the example of FIG. 12, the region of, for example, the portal vein is automatically detected from the MR 3D image data. By referring to this region, the corresponding cross section is displayed in the real-time ultrasonic tomographic image, while the ultrasonic probe 70 is being moved.

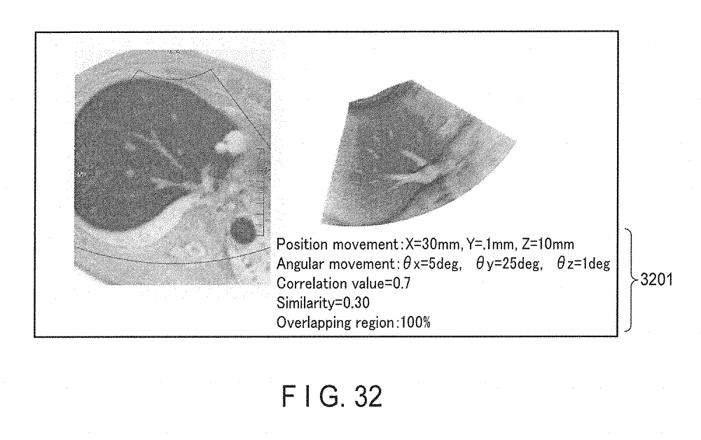

FIG. 31 and FIG. 32 illustrate embodiments in which alignment results are displayed. FIG. 31 illustrates an embodiment of quality 3101 of alignment between 3D ultrasonic image data. FIG. 32 illustrates an embodiment of quality 3201 of alignment between 3D medical image data and 3D ultrasonic image data. Position movement amounts and angular movement amounts relative to the reference volume by the image alignment calculation illustrated in FIG. 4 are displayed. When a mutual information amount (MI value) is used as a similarity function of alignment, the MI value is displayed. Alternatively, independently from the similarity function of alignment, a similarity of images, such as a brightness difference value of images, is displayed. The ratio of the overlapping region between 3D image data before alignment or after alignment is displayed. Since the region of the 3D ultrasonic image is small, the overlapping amount greatly affects the quality of alignment.

Thereby, the doctor can obtain information relating to the quality of alignment, etc. By the doctor's judgment, based on the quality information, it is thinkable to cancel the alignment process, or to retry the alignment process by changing conditions.

Furthermore, the following function of the system is thinkable. The system prepares, in advance, algorithms of judgment for the position movement amount, angular movement amount, an evaluation value of a similarity function of alignment, a similarity of images, and the amount or ratio of the overlapping region between 3D medical image data. When the range of the set reference is exceeded, the system automatically cancels the alignment process.

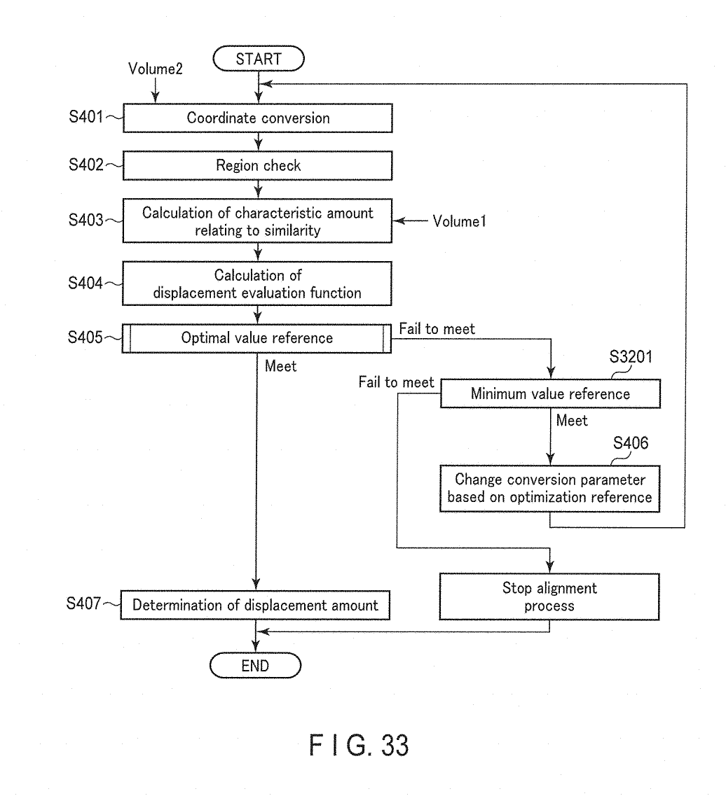

FIG. 33 illustrates another example of the flowchart of the process illustrated in FIG. 4. Specifically, in step S3201, it is judged whether or not to meet a set reference (minimum value reference) for tolerating the alignment result. As the set reference for tolerating the alignment result, for example, the following conditions may be set: “movement distance<**mm or less”, “rotation amount<**degrees or less”, “similarity function value<**or more”, “image similarity<**or more”, and “overlap ratio<**or more”.

As the similarity function, various evaluation functions, such as a mutual information amount and a cross-correlation amount, are thinkable. As the image similarity, various evaluation functions, such as a brightness difference value, are thinkable.

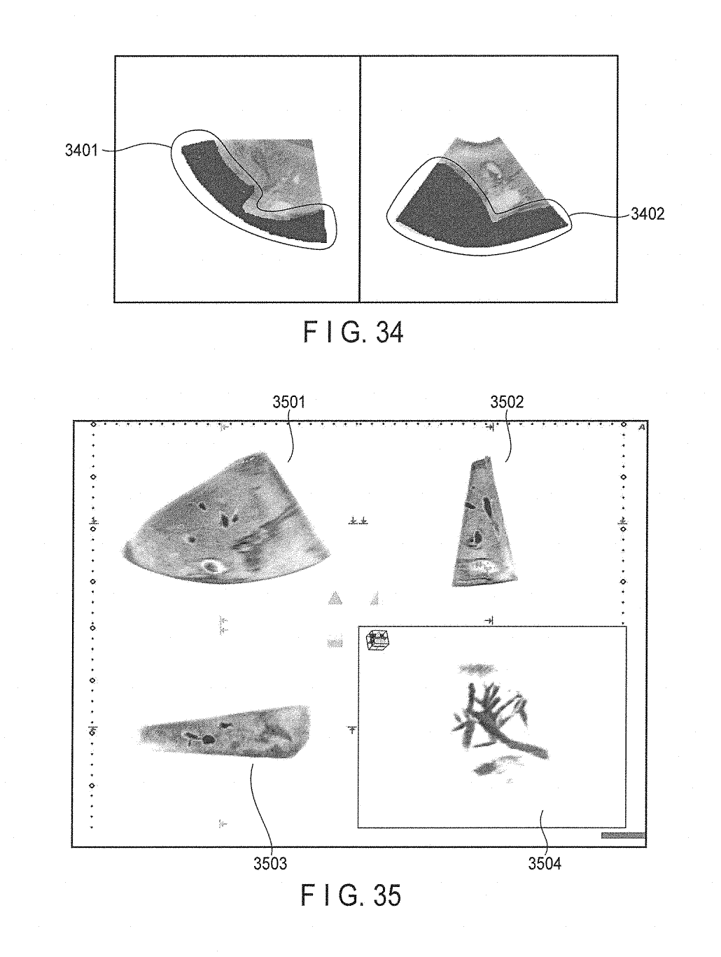

The control circuitry 23, which executes the image alignment function, may additionally include a function of detecting a noise region in the 3D medical image data or ultrasonic image data, and excluding the noise region from the alignment calculation. FIG. 34 illustrates an embodiment of 3D ultrasonic image data. A 3D ultrasonic image before a treatment is displayed on the left side of the monitor, and a 3D ultrasonic image after the treatment is displayed on the right side of the monitor.

In the ultrasonic images illustrated in FIG. 5, a noise region 3401 and a noise region 3402 are defined by desired conditions, and noise regions are extracted by image processing. The detected noise region 3401 and noise region 3402 are excluded from the image alignment calculation. In an example of an algorithm for extracting the noise region 3401 and noise region 3402, the level of a brightness value or the dispersion of a brightness value is thinkable as an index. In addition, as regards the ultrasonic image, transmission of an ultrasonic signal is not executed, and a similar 3D image is generated by only the reception and is set as a 3D image of a noise image. The 3D image data, with respect to which the ultrasonic is transmitted and received, and the 3D image data of the noise image are compared with respect to a brightness difference or the like, and a similar region can be defined as a noise region. In accordance with the image alignment process, the noise region is excluded, and thereby the precision of alignment is improved. When the alignment between the 3D medical image and 3D ultrasonic image is executed, it is thinkable that only the 3D ultrasonic image is excluded from the above-described calculation of the noise region.

It is thinkable that the control circuitry 23, which executes the image alignment function 105, detects a region having a common structure in the 3D medical image data or ultrasonic image data, and executes the image alignment calculation. In the image alignment, a blood vessel structure is an important alignment structure.

As illustrated in FIG. 35, 3D ultrasonic color data 3501, 3502 and 3503 are MPR display, and blood vessel regions are extracted by a Doppler method. In the alignment between 3D ultrasonic image data, it is thinkable to execute alignment between 3D ultrasonic color data. In the alignment between the CT 3D data or MR 3D data, and the 3D ultrasonic image data, the hepatic vein or portal vein can be extracted in the CT or MR by a desired segmentation process. Image alignment between extracted blood vessels is thinkable. Also in the 3D ultrasonic image data, the segmentation process is executed with respect to vascular cavities, based on brightness values or the like, and the vascular cavities can be used for image alignment. It is also thinkable that the segmentation process is executed on contrast ultrasonic data 3504 in which blood flow information is emphasized.

Although the flowchart illustrated in FIG. 3 was described in connection with the case of the alignment process between ultrasonic image data, this flowchart may be applied to an alignment process between the ultrasonic image data and medical image data by other modalities.

Furthermore, the process of correcting a displacement due to a body motion or respiratory time phase, which is illustrated in FIG. 7, is not limited to the alignment between ultrasonic image data, and is also applicable to an alignment process between ultrasonic image data and medical image data by other modalities.

The term “processor” used in the above description means, for example, a CPU (Central Processing Unit), a GPU (Graphics Processing Unit), or circuitry such as an ASIC (Application Specific Integrated Circuit), or a programmable logic device (e.g. SPLD (Simple Programmable Logic Device), CLPD (Complex Programmable Logic Device), FPGA (Field Programmable Gate Array)). The processor realizes functions by reading out and executing programs stored in the storage circuitry. In the meantime, each processor of the embodiments is not limited to the configuration in which each processor is configured as single circuitry. Each processor of the embodiments may be configured as a single processor by combining a plurality of independent circuitries, thereby to realize the function of the processor. Furthermore, a plurality of structural elements in FIG. 1 may be integrated into a single processor, thereby to realize the functions of the structural elements.

In the above description, the case is assumed in which the alignment between the ultrasonic image data and medical image data is the alignment between two data. However, the alignment between three or more data may be executed. For example, currently scanned ultrasonic image data, previously captured ultrasonic image data, and CT 3D image data may be aligned and displayed in parallel.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasonic diagnostic apparatus comprising
processing circuitry configured to:
acquire position information relating to an ultrasonic probe and an ultrasonic image;
acquire ultrasonic image data which is obtained by transmission and reception of ultrasonic from the ultrasonic probe at a position where the position information is acquired, the ultrasonic image data being associated with the position information;
execute associating between a first coordinate system relating to the position information and a second coordinate system relating to medical image data; and
execute image alignment between an ultrasonic image based on the associated ultrasonic image data and a medical image based on the medical image data.

2. The apparatus according to claim 1, wherein the processing circuitry is further configured to determine region information which serves as a reference for the image alignment, in at least one of the ultrasonic image and the medical image,
wherein the processing circuitry executes the image alignment based on the region information.

3. An ultrasonic diagnostic apparatus comprising
processing circuitry configured to:
acquire position information relating to an ultrasonic probe and an ultrasonic image;
execute associating between a first coordinate system relating to the position information and a second coordinate system relating to medical image data;
determine region information which serves as a reference for image alignment, in at least one of an ultrasonic image based on the first coordinate system and a medical image based on the second coordinate system;
acquire ultrasonic image data which is obtained by transmission and reception of ultrasonic from the ultrasonic probe at a position where the position information is acquired, the ultrasonic image data being associated with the position information; and
execute, based on the associating and the region information, image alignment between an ultrasonic image based on the ultrasonic image data and the medical image.

4. An ultrasonic diagnostic apparatus comprising
processing circuitry configured to:
acquire ultrasonic image data;
determine region information which serves as a reference for image alignment, in at least one of an ultrasonic image based on the ultrasonic image data and a medical image based on medical image data; and
execute, based on the region information, image alignment between the ultrasonic image and the medical image.

5. The apparatus according to claim 1, wherein the processing circuitry is further configured to synchronously display, based on a relationship between the first coordinate system and the second coordinate system which is determined by completion of the image alignment, a real-time ultrasonic image based on ultrasonic image data newly acquired by the ultrasonic probe, and a medical image based on the medical image data corresponding to the real-time ultrasonic image.

6. The apparatus according to claim 1, wherein the position information is acquired by a position sensor system which utilizes at least one of a magnetic sensor, an infrared sensor, an image recognition process, a gyro sensor and a robotic arm.

7. The apparatus according to claim 1, wherein the processing circuitry associates the first coordinate system with the second coordinate system at a position where a target region included in the medical image data is depicted on the ultrasonic image by the ultrasonic probe being operated.

8. The apparatus according to claim 1, wherein when the medical image data is ultrasonic image data acquired by a common coordinate system to the first coordinate system, the processing circuitry executes the associating by using the common coordinate system.

9. The apparatus according to claim 1, wherein the processing circuitry sets, as an origin of the first coordinate system, a point where a sensor is attached on a body surface of a living body, or a point designated on the body surface of the living body.

10. The apparatus according to claim 1, wherein the ultrasonic image data is at least one of two-dimensional image data, three-dimensional image data and four-dimensional image data.

11. The apparatus according to claim 10, wherein the three-dimensional image data is data acquired by reconstructing two-dimensional image data with which the position information is associated.

12. The apparatus according to claim 10, wherein the three-dimensional image data and the four-dimensional image data are data in which position information is associated with three-dimensional image data acquired by electronic scan by a mechanical four-dimensional probe or a two-dimensional array probe.

13. The apparatus according to claim 5, wherein the ultrasonic image data is three-dimensional image data with which the position information is associated, or four-dimensional image data with which the position information is associated, and
the processing circuitry synchronously displays, a three-dimensional real-time ultrasonic image, and a corresponding cross section of a three-dimensional medical image which corresponds to the three-dimensional real-time ultrasonic image.

14. The apparatus according to claim 2, further comprising a user interface which prompts a user to determine, as the region information, corresponding points or corresponding regions between the ultrasonic image and the medical image,
wherein the processing circuitry corrects, based on the region information, the associating between the first coordinate system and the second coordinate system.

15. The apparatus according to claim 3, further comprising a user interface which prompts a user to determine a desired region in the medical image data, and prompts the user to determine a corresponding region which corresponds to the desired region in a cross-sectional image of real-time ultrasonic image data,
wherein the processing circuitry corrects the associating between the first coordinate system and the second coordinate system, based on coordinates of the corresponding region, and newly acquires ultrasonic image data, based on the corrected associating.

16. The apparatus according to claim 14, further comprising display processing circuitry configured to display the ultrasonic image and the medical image,
wherein the processing circuitry supports an input of the corresponding regions to the ultrasonic image and the medical image which are displayed.

17. The apparatus according to claim 14, further comprising display processing circuitry configured to display the ultrasonic image and the medical image in parallel,
wherein the ultrasonic image is acquired after the ultrasonic image and the medical image are displayed in parallel.

18. The apparatus according to claim 2, wherein the processing circuitry refers to a database which stores a two-dimensional image pattern or a three-dimensional image pattern of a region serving as a landmark, and detects a region corresponding to a determined landmark from each of the ultrasonic image data and the medical image data.

19. The apparatus according to claim 1, wherein the processing circuitry displays at least one of a displacement estimation amount by a calculation result of alignment, an evaluation value of a similarity function of the alignment, a similarity between images, and an amount or a ratio of overlapping between data.

20. The apparatus according to claim 1, wherein the processing circuitry controls the image alignment, based on a displacement amount obtained as a calculation result of the image alignment.

21. The apparatus according to claim 1, wherein the processing circuitry controls the image alignment, based on a similarity obtained as a calculation result of the image alignment.

22. The apparatus according to claim 1, wherein when the medical image data is ultrasonic image data, the processing circuitry controls the image alignment, based on a degree of overlapping between ultrasonic image data in a three-dimensional space.

23. The apparatus according to claim 1, wherein the processing circuitry extracts a noise region from each of the ultrasonic image data and the medical image data, and executes a calculation of the image alignment by excluding the noise region.

24. The apparatus according to claim 1, wherein the processing circuitry extracts a region with a common structure from each of the ultrasonic image data and the medical image data, and uses the region with the common structure in a calculation of the image alignment.

25. An ultrasonic diagnostic method comprising:
acquiring position information relating to an ultrasonic probe and an ultrasonic image;
acquiring ultrasonic image data which is obtained by transmission and reception of ultrasonic from the ultrasonic probe at a position where the position information is acquired, the ultrasonic image data being associated with the position information;
executing associating between a first coordinate system relating to the position information and a second coordinate system relating to medical image data; and
executing image alignment between an ultrasonic image based on the associated ultrasonic image data and a medical image based on the medical image data.

26. The method according to claim 25, further comprising determining region information which serves as a reference for the image alignment, in at least one of the ultrasonic image and the medical image,
wherein the executing executes the image alignment based on the region information.

27. An ultrasonic diagnostic method comprising:
acquiring position information relating to an ultrasonic probe and an ultrasonic image;
executing associating between a first coordinate system relating to the position information and a second coordinate system relating to medical image data;
determining region information which serves as a reference for image alignment, in at least one of an ultrasonic image based on the first coordinate system and a medical image based on the second coordinate system;
acquiring ultrasonic image data which is obtained by transmission and reception of ultrasonic from the ultrasonic probe at a position where the position information is acquired, the ultrasonic image data being associated with the position information; and
executing, based on the associating and the region information, image alignment between an ultrasonic image based on the ultrasonic image data and the medical image.

28. An ultrasonic diagnostic method comprising:
acquiring ultrasonic image data;
determining region information which serves as a reference for image alignment, in at least one of an ultrasonic image based on the ultrasonic image data and a medical image based on medical image data; and
executing, based on the region information, image alignment between the ultrasonic image and the medical image.
